(19) United States
(12) Patent Application Publication
(10) Pub. No.: US 2023/0371517 A1
(43) Pub. Date: Nov. 23, 2023

(54) COMBINATIONS TREATMENTS

(71) Applicant: Aequor, Inc., Encinitas, CA (US)

(72) Inventor: Cynthia K. Burzell, Encinitas, CA (US)

(73) Assignee: Aequor, Inc., Encinitas, CA (US)

(21) Appl. No.: 18/248,020

(22) PCT Filed: Oct. 12, 2021
(86) PCT No.: PCT/US2021/054611

§ 371 Date: Apr. 5, 2023

Related U.S. Application Data
(60) Provisional application No. 63/090,957, filed on Oct. 13, 2020.

(51) Int. Cl. A01N 57/28; A01N 59/16; A01P 1/00

(52) U.S. Cl. CPC A01N 57/28; A01N 59/16; A01P 1/00

(57) ABSTRACT

This disclosure relates to organophosphorous and organosulfurous compounds and their use in the amelioration, treatment, and/or prevention of microbial infections and diseases in a subject. The disclosure also relates to combination therapy using a composition comprising organophosphorous compounds, organosulfurous compounds, and antimicrobials in methods for treating or inhibiting microbial infections and diseases.

### CROSS REFERENCE TO RELATED APPLICATIONS

This application claims the benefit of U.S. Provisional Application No. 63/090,957 filed Oct. 13, 2020, entitled “Antimicrobial Combination Therapeutics”, which is incorporated herein by reference in its entirety.

### FIELD

This disclosure relates to organophosphorous and organosulfurous compounds and salts, acids, and bases thereof and their use in the amelioration, treatment, or prevention of microbial infections and diseases in a human, animal, and/or other living organisms. The disclosure also relates to therapies using a composition comprising one or more of organophosphorous compounds, organosulfurous compounds, or salts, acids or bases thereof or combination therapies using the composition described herein in combination with one or more antimicrobials, one or more antimicrobial potentiators, and/or one or more prebiotics in methods for treating or inhibiting microbial infections and diseases.

### BACKGROUND

The development of antimicrobial-resistant (AMR) strains of bacteria, fungi, viruses, and other microorganisms have led to hard-to-treat infections and diseases. The 3 types of antibiotic resistance are: acquired, intrinsic, and adaptive. These resistance responses have led to antimicrobial-resistant strains of microorganisms. In addition, bacteria, fungi, viruses, and other microorganisms produce biofilms that allow them to adhere to surfaces and excrete an extracellular matrix that offers protection as they colonize. Biofilm is the natural response mechanism to shield the bacterial and fungi from environmental stresses, such as heat and other forms of sterilization, the human and animal immune systems, antimicrobial biocides, fungicides, antivirals, and antibiotics. As a multi-layered defense, biofilm adapts and builds, sloughs off in air and water, and spreads the pathogens to new surfaces where they re-activate and form new colonies and biofilms. Biofilm-specific resistance is multifactorial as there are multiple mechanisms that contribute to resistance. For example, the biofilm barrier prevents antibody penetration. As another example, inside the biofilm, pathogens enter a dormant state as persister cells that are phenotypic variants resistant to antibiotics. Medically relevant biofilms are associated with contamination on inert, nano, and organic surfaces, non-critical and critical clinical surfaces, indwelling medical devices, and tissues and organs of humans, animals, and/or other living organisms. The U.S. Centers for Disease Control (CDC) associate biofilm with 90% of hospital-acquired infections and 80% of chronic infections. The World Health Organization (WHO) published a list of 12 priority pathogens, all of which are biofilm-formers, for which new antibiotics and other remedies are urgently needed, including Staphylococcus aureus methicillin-sensitive (MSSA), methicillin-resistant Staphylococcus aureus (MRSA), vancomycin-resistant Staphylococcus aureus (VRSA), Acinetobacter baumannii, Pseudomonas aeruginosa, and Enterobacteriaceae (e.g., Salmonella enterica and E. coli). All bacterial and fungal pathogens on the CDC lists of urgent, pandemic, and biothreats threats are also biofilm-formers. As an example, MRSA is resistant to β-lactams, such as methicillin. For most MRSA strains, vancomycin is the only effective antibiotic agent today. However, biofilm-forming MRSA strains with intermediate-resistance resistance to vancomycin (VISA) and resistance to vancomycin (VRSA) have been reported. Biofilms complicate the treatment of infections and diseases. Thus, there is a need for novel therapies to treat biofilm-forming antibiotic and non-antibiotic resistant strains.

The body's bacterial, fungal, and viral inhabitants (i.e., microbiome), play key roles in health and disease. For example, the skin microbiome protects against skin infection by outcompeting pathogenic organisms. An imbalanced skin microbiome can be associated with many diseases, poor wound healing, and accelerated skin aging. There is a need for novel therapies to prevent the growth of pathogenic organisms and promote the growth of the microbiome and improve skin health.

### SUMMARY

Described herein is a method of treating or inhibiting a microbial infection in a human, animal, or other living organisms, the method comprising administrating an effective amount of a composition comprising one or more of an organophosphorous compound, organosulfurous compound, or a salt, an acid, or a base thereof. In embodiments, the composition further comprises one or more antimicrobials (including but not limited to antibacterial/antibiotic, antiviral, antifungal), one or more antimicrobial potentiators, and/or one or more prebiotics.

In embodiments, the organophosphorous compound, organosulfurous compound, or a salt, an acid, or a base thereof is in the form of nanoparticles.

In embodiments, administration of the composition is topical, oral, intravenous, aerosolized, and/or other routes of administration.

In embodiments, the microbial infection is pathogenic to humans, animals, and/or other living organisms.

The infection or disease is caused by one or more microorganisms, such as bacteria, fungi, and viruses. In embodiments, the one or more microorganisms are bacteria. Examples of bacteria include Staphylococcus aureus, Staphylococcus epidermidis, Acinetobacter baumannii, Pseudomonas aeruginosa, Enterobacteriaceae, Escherichia coli, Salmonella spp., Salmonella bongori, Enterococcus faecium, Helicobacter pylori, Campylobacter spp., Neisseria gonorrhoeae, Streptococcus pneumoniae, Streptococcus mutans, Streptococcus gordonii, Streptococcus pyogenes, Haemophilus influenzae, Shigella spp., Klebsiella pneumoniae, Clostridium difficile, Bacillus anthracis, Yersinia pestis, Francisella tularensis, Burkholderia mallei, Burkholderia pseudomallei, Corynebacterium spp., Micrococcus luteus, Micrococcus lylae, Micrococcus roseus, Cutibaceterium acnes, Vibrio vulnificus, Vibrio cholerae, Vibrio parahaemolyticus, Propionibacterium spp., Neisseria gonorrhoeae, Burkholderia cepacia, Burkholderia mallei, Burkholderia pseudomallei, Ralstonia pickettii, Cuprividus metallidurans and/or other biofilm-forming bacteria.

In embodiments, the one or more microorganisms are viruses. Examples of viruses include including influenza virus, rhinovirus, coronavirus, respiratory syncytial virus, parainfluenza, norovirus, human immunodeficiency virus, dengue virus, pox viruses, herpes virus, adenoviruses, parvoviruses, paramyxovirus, tobacco mosaic virus, flavivirus, rotavirus, and/or other biofilm-associated viruses.

In embodiments, the one or more microorganisms are fungi. Examples of fungi include Candida albicans, Candida auris, Aspergillus fumigatus, Aspergillus flavus, Apohysomyces sp., Blastomyces dermatitides, Coccidioides posadasii, Cryptococcus neoformans, Fusarium spp., Histoplasma capsulatum, Pneumocystis firovecii, Rhizopus oryzae, Scedosporium spp., and/or other biofilm-forming fungi.

In embodiments, the infection is a bacterial or fungal infection that is methicillin-sensitive, methicillin-resistant, vancomycin-intermediate, vancomycin-resistant, carbapenem-resistant, fluoroquinolone-resistant, clarithromycin-resistant, cephalosporin-resistant, ampicillin-resistant, penicillin-non-susceptible, fluconazole-resistant, amphotericin-resistant, echinocandins-resistant, mupirocin-resistant, erythromycin-resistant, streptomycin-resistant, tetracycline-resistant, amoxicillin-resistant, ciprofloxacin-resistant, silver-resistant, salicylic acid-resistant, neomycin sulfate-resistant, polymyxin B sulfate-resistant, bacitracin-resistant, zinc-resistant, alcohol-tolerant, alcohol-resistant, or chlorhexidine-resistant.

In embodiments, the organophosphorous or organosulfurous compounds, or salts, acids, or bases thereof are sodium dodecylbenzenesulfonate (SDBS), sodium dodecyl sulfate (SDS), 4-Dodecylbenzenesulfonic acid (DBSA), butyl phosphoramidate (BPA), or (4-aminophenethyl)dimethylphosphine oxide (APDMPO).

In embodiments, the organophosphorous compound, organosulfurous compound, or a salt, an acid, or a base thereof is present in the composition at a concentration of 0.01 μg/ml to 300 μg/ml.

The composition described herein is administered with one or more additional agents such as one or more antimicrobials, antimicrobial potentiators, and/or probiotics. In embodiments, the composition described herein can include one or more antimicrobials, antimicrobial potentiators, and/or probiotics.

Examples of antimicrobial include β-lactams, aminoglycosides, glycopeptides, macrolides, fluoroquinolones, sulfonamides, tetracyclines, mupirocin, salicylates, polymixins, butenafine hydrochloride, clotrimazole, miconazole nitrate, terbinafine hydrochloride, fluoroquinolone carboxylic acid derivatives, tryptophan, thiosulfil, plazomicin, fosfomycin, cefepime, maxipime, pravibismane, and tolnaftate. In embodiments, the antimicrobial includes an antibiotic such as penicillin, oxacillin, gentamicin, vancomycin, piperazines, bismuth thiols, bismuth thiol complexes, bismuth dithiol complexes, chlorhexidine, itaconic acid, colistin, potassium carbonate, isothiocyanates, phenyl isothiocyanate, sodium carbonate, sodium bicarbonate, calcium phosphate, calcium carbonate, and methyl ethyl ketone. In embodiments, the concentration of the antimicrobial administered is a concentration of 0.01 μg/ml to 3 μg/ml. In embodiments, the antimicrobial is in the form of nanoparticles.

In embodiments, the composition described herein includes one or more antiseptics. In embodiments, the antiseptic includes iodine, povidone-iodine, acetic acid, hydrogen peroxide, peroxide, peracetic acid, and sodium hypochlorite. In embodiments, the concentration of the antiseptic administered is a concentration of 0.01% to 30%. In embodiments, the antiseptic is in the form of nanoparticles.

In embodiments, the composition described herein includes one or more antimicrobials, and one or more of a silver compound, zinc compound, salicylic acid, benzoyl peroxide, and/or plant, animal, or chemically derived compound. In embodiments, the silver compound is silver ions, silver particles, silver nanoparticles, metallic silver, colloidal silver, or silver chloride. In embodiments, the concentration of the silver is 0.01 ppm to 30 ppm. In embodiments, the zinc compound includes zinc acetate, zinc octoate, zinc pyrithione, zinc sulfate, or zinc oxide. In embodiments, the concentration of zinc is 0.01% to 5%. In embodiments, the concentration of salicylic acid is 0.01% to 5%. In embodiments, the concentration of benzoyl peroxide is 1% to 10%.

In embodiments, the plant, animal, or chemically derived compound includes orange essential oil, lemon essential oil, oregano essential oil, thyme essential oil, cinnamon essential oil, citral, cinnamaldehyde, carvacrol, thymol, alpha-hydroxy acid, glycolic acid, malic acid, maleic acid, clavulanic acid, glucaric acid, formic acid, hydrochloric acid, hypochlorous acid, jasmonic acid, giant knotweed extract, amino acids, L-lysine, tryptophan, and/or aloe. In embodiments, the concentration of the plant, animal, or chemically derived compound is 0.01% to 5%. In embodiments, the plant, animal, or chemically derived compound is in the form of nanoparticles.

The composition described herein includes one or more of antibiotic adjuvant β-lactamase inhibitors, penicillin-binding protein inhibitors, dihydropteroate synthetase inhibitors, or iron chelators. In embodiments, the β-lactamase inhibitor is taniborbactam. In embodiments, the iron chelator is deferiprone, desferasirox, desferoxamine mesylate, or FR¹⁶⁰. In embodiments, the concentration of antibiotic adjuvant is 0.01 μg/ml to 500 μg/ml. In embodiments, the antibiotic adjuvant is in the form of nanoparticles.

The composition described herein includes one or more non-steroidal anti-inflammatory drugs. In embodiments the non-steroidal anti-inflammatory drug is aspirin. In embodiments, the non-steroidal anti-inflammatory drug is tocilizumab. In embodiments, the concentration of aspirin is 0.01 μg/ml to 50 μg/ml. In embodiments, the non-steroidal anti-inflammatory drug is in the form of nanoparticles.

The composition described herein includes one or more antihistamines. In embodiments, the antihistamine drug is an H₁-antihistamines, H₂-antihistamines, H₃-antihistamines, and H₄-antihistamines. In embodiments, the H₂-antihistamines famotidine. In embodiments, the concentration of the antihistamine is 0.01 μg/ml to 100 μg/ml. In embodiments, the antihistamine is in the form of nanoparticles.

The composition described herein includes one or more antibiofilm agents. In embodiments, the antibiofilm agent interferes with bacterial signals, interferes with bacterial systems, interferes with the physical attachment, and induces bacterial cells to detach.

In embodiments, the antibiofilm agent includes one or more bacterial signaling system inhibitors, such as protein kinase inhibitors, thioredoxin reductase inhibitors, and quorum sensing inhibitors. In embodiments, the protein kinases inhibitors include serine/threonine kinase inhibitors, acetate kinase inhibitors, tyrosine kinase inhibitors, and serine/threonine/tyrosine kinase inhibitors. In embodiments, the protein kinases inhibitor is an isothiocyanate. In embodiments, the quorum sensing signal inhibitor includes, Avellanin C, p-nitrophenyl glycerol, tannic acid, isothiocyanate, bismuth thiol, bismuth thiol complexes, bismuth thiol complexes, bismuth dithiol complexes, and RNAIII inhibiting peptides. In embodiments, the concentration of protein kinase inhibitors and quorum sensing inhibitors is 0.01 μg/ml to 500 μg/ml. In embodiments, the compounds that interfere with physical attachment of the cells to surfaces include compounds that interfere with adhesins, pili, and extracellular polymeric matrices. In embodiments, the compounds that induce the bacterial cells to detach include sugars, amino acids, carbon sources, salt, and other nutrients. In embodiments, the bacterial signaling system inhibitor is in the form of nanoparticles.

Microbially produced compounds such as metabolites, bacteriocins, enzymes, and acids have dual roles to inhibit the invasion of other species and promote metabolism and immunity. In embodiments, the composition further comprises microbially produced compounds such as metabolites, bacteriocins, enzymes, and acids. In embodiments, the microbially produced compounds include, propionate, butyrate, acetate, serine endopeptidase, succinic acid, lactic acid, formic acid, propionic acid, itaconic acid, lysozyme, phospholipases, phospholipase A2, defensins, cathelicidins, lactotransferrin, transferrin, hydrogen peroxide, nisin, actagardine, durancin 61A, and PsVP-10. In embodiments, the concentration of microbially produced metabolite, bacteriocin, enzyme, and acid is 0.01 μg/ml to 500 μg/ml and 1% to 10% (v/v).

In compositions described herein includes light therapy. In embodiments, the light is UVC, UVB, UVA, blue, and infrared wavelengths. In embodiments, the light has a minimum average power density of 0.1 w/cm² to 10 w/cm². In embodiments, the duration of treatment is 1 minute to 120 minutes.

In embodiments, the combination treatment is administered together.

In embodiments, the combination treatment is administered in tandem.

Further described herein is a method of preventing the growth of and/or removing or killing microorganisms on or in a critical clinical surface, such as a medical device, the method comprising contacting a critical clinical surface with an effective amount of a composition comprising one or more of an organophosphorous compound, organosulfurous compound, or a salt, acid, or base thereof. In embodiments, the composition further comprises one or more antimicrobials (including but limited to an antibacterial/antibiotic, antiviral, or antifungal), one or more antimicrobial potentiators, and/or one or more probiotics.

In embodiments, the composition is a cleaning agent, dispersant, surfactant, anti-odor agent, antibiofilm agent (including but not limited to biofilm removal agent, biofilm disruption agent, biofilm inhibition agent, etc.), antifouling agent (including but not limited to fouling removal agent, fouling disruption agent, fouling inhibition agent), antimicrobial growth agent (including but not limited to disrupt, remove, prevent, and/or inhibit microbial growth), an agent for killing microorganisms (including bacteria, fungi, viruses, and other microorganisms), a therapeutic or a prophylactic antibiotic, or a combination thereof.

### BRIEF DESCRIPTION OF THE DRAWINGS

FIGS. 1A and 1B show biofilm inhibition by butyl phosphoramidate (BPA), sodium dodecylbenzenesulfonate (SDBS), and 4-Dodecylbenzenesulfonic acid (DBSA) against Staphylococcus aureus ATCC 12600 and E. coli ATCC 11775, which was determined by (A) measuring total intensity with Ex/Em (nm): 456/541 and (B) staining biofilm with Syto 9 green-fluorescent nucleic acid stain. Biofilm images appear white and gray and images without biofilm appear black. Images of untreated biofilm, sub-MBIC, and MBIC were taken using MiniMax 300 Imaging Cytometer (Molecular Devices).

FIG. 2 shows the minimum biofilm eradicating concentration (MBEC) of butyl phosphoramidate (BPA) against S. aureus ATCC 6358. Colony counting of viable cells of quadruplicates in CFU/ml after 10 minutes of exposure to BPA demonstrated 99.99% removal of biofilm. Data corresponds to 4.4-Log reduction.

### DETAILED DESCRIPTION

This disclosure describes compositions comprising one or more organophosphorous, and organosulfurous compounds, or salts, acids, or base thereof, and their use in the amelioration, treatment, or prevention of fungal and infections and diseases caused by a microorganism in a subject. In embodiments, the compositions described herein further comprise one or more antimicrobials, antimicrobial potentiators, and/or prebiotics.

The disclosure also describes therapy using the compositions described herein and combination therapies for treating or inhibiting microbial infections and diseases. In embodiments, the compositions for combination therapies comprise one or more organophosphorous, and organosulfurous compounds, or salts, acids, or bases thereof, and one or more additional agents such as one or more of antimicrobials, antimicrobial potentiators, and/or prebiotics.

Methods described herein include treating subjects (humans, veterinary animals (dogs, cats, reptiles, birds, etc.), livestock (horses, cattle, goats, pigs, chickens, etc.), and research animals (monkeys, rats, mice, fish, etc.). Subjects in need of a treatment (in need thereof) are subjects suffering from or diagnosed with a microbial infection.

As used herein, the term “or” is understood to be inclusive unless specifically stated or obvious from context to the contrary. As used herein, the terms “a”, “an”, and “the” are understood to be singular or plural unless specifically stated or obvious from context to the contrary.

Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; and A (alone); B (alone); and C (alone).

As used herein, the term “antimicrobial” refers to an agent that is effective against pathogenic microorganisms, including bacteria, fungi, viruses, protozoa, and biofilms. Antimicrobial agents can be used to disperse, remove, inhibit, reduce, or prevent microbial growth.

The term “heteroatom” refers to any atom other than carbon, for example, N, O, or S.

The term “substituents” refers to groups such as hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitro, amino, amido, aldehyde, acyl, oxyacyl, carboxyl, sulfonyl, sulfonamide, sulfuryl, and the like.

The term “hydrocarbyl” refers to univalent groups formed by removing a hydrogen atom from a hydrocarbon, e.g. alkyl, cycloalkyl, alkenyl, alkynyl, aryl, alkylaryl, arylalkyl, arylalkenyl, arylalkynyl, and arylene. The term “substituted hydrocarbyl” refers to hydrocarbyl groups further bearing one or more substituents as defined herein.

The term “alkyl” refers to a monovalent straight or branched chain hydrocarbon group having from one to 12 carbon atoms, for example, methyl, ethyl, n-propyl, iso-propyl, n-butyl, iso-butyl, tert-butyl, n-pentyl (also known as n-amyl), n-hexyl, and the like. The term “substituted alkyl” refers to alkyl groups further bearing one or more substituents as defined herein.

The term “alkenyl” refers to straight-chained or branched hydrocarbyl groups having at least one carbon-carbon double bond and having 2 to 12 carbon atoms, and the term “substituted alkenyl” refers to alkenyl groups further bearing one or more substituents as defined herein.

The term “alkynyl” refers to straight-chained or branched hydrocarbyl groups having at least one carbon-carbon triple bond, and having 2 to 12 carbon atoms, and the term “substituted alkynyl” refers to alkynyl groups further bearing one or more substituents as defined herein.

The term “alkoxy” refers to the moiety —O-alkyl, wherein alkyl is as defined above, and the term “substituted alkoxy” refers to alkoxy groups further bearing one or more substituents as defined herein.

The term “cycloalkyl” refers to alkyl groups having 3 and 8 carbon atoms arranged as a ring, and the term “substituted cycloalkyl” refers to cycloalkyl groups further bearing one or more substituents as defined herein.

The term “aromatic” refers to a cyclically conjugated molecular entity with a stability, due to derealization, significantly greater than that of a hypothetical localized structure, such as the Kekule structure.

The term “heterocyclic,” when used to describe an aromatic ring, refers to the aromatic rings containing at least one heteroatom, as defined above. The term “heterocyclic,” when not used to describe an aromatic ring, refers to cyclic (i.e., ring-containing) groups other than aromatic groups, the cyclic group being formed by 3 and 14 carbon atoms and at least one heteroatom as defined herein.

The term “substituted heterocyclic” refers, for both aromatic and non-aromatic structures, to heterocyclic groups further bearing one or more substituents as defined herein.

The term “aryl” refers to aromatic groups having 5 to 14 carbon atoms and the term “substituted aryl” refers to aryl groups further bearing one or more substituents as defined herein.

The term “heteroaryl” refers to aromatic rings, where the ring structure is formed by 3 to14 carbon atoms and by at least one heteroatom described above, and the term “substituted heteroaryl” refers to heteroaryl groups further bearing one or more substituents as defined herein.

The term “alkylaryl” refers to alkyl-substituted aryl groups and the term “substituted alkylaryl” refers to alkylaryl groups further bearing one or more substituents as defined herein.

The term “arylalkyl” refers to aryl-substituted alkyl groups and the term “substituted arylalkyl” refers to arylalkyl groups further bearing one or more substituents as defined herein.

The term “arylalkenyl” refers to aryl-substituted alkenyl groups and the term “substituted arylalkenyl” refers to arylalkenyl groups further bearing one or more substituents as defined herein.

The term “arylalkynyl” refers to aryl-substituted alkynyl groups and the term “substituted arylalkynyl” refers to arylalkynyl groups further bearing one or more substituents as defined herein.

The term “arylene” refers to divalent aromatic groups having 5 to 14 carbon atoms and the term “substituted arylene” refers to arylene groups further bearing one or more substituents as defined herein.

The present disclosure describes a method for treating or inhibiting a fungal or bacterial infection in a patient, the method comprising administrating a composition in an effective amount comprising one or more of compounds described herein, such as an organophosphorous compound, organosulfurous compound, and an additional agent such as an antimicrobial, an antimicrobial potentiator, and/or a probiotic.

In embodiments, the compounds described herein are represented by Formula I, or a salt, acid, or base thereof:

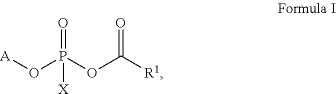

wherein A is C₁₋₁₀ hydrocarbyl or C₁₋₁₀ hydrocarbyl substituted with R¹; X is NHR, NHOR, NHCOR, NHOCOR, or OR; and R is H or C₁₋₁₀hydrocarbyl;
R¹ is hydrogen, halogen, cyano, OH, C₁₋₆ hydrocarbyl, C₁₋₆ alkoxy, SOR², SO₂R², SO₂NR³R⁴COR², CO₂R², CONR³R⁴, NR³R⁴, NR³COR⁴, NR³SO₂R⁴, NR³CO₂R⁴, NR³CONR⁴, OCOR², or phosphonic acid, wherein each of C₁₋₆ hydrocarbyl, C₁₋₆ alkoxy, SOR², SO₂R², SO₂NR³R⁴, COR², CO₂R², CONR³R⁴, NR³R⁴, NR³COR⁴, NR³SO₂R⁴, NR³CO₂R⁴, NR³CONR⁴, or OCOR², can be optionally substituted with halo, amino, hydroxyl, C₁₋₆ hydrocarbyl, C₁₋₆ alkoxy, cyano, or phosphonic acid; and
R², R³, and R⁴ are independently selected from hydrogen or C₁₋₆ hydrocarbyl, wherein each of the C₁₋₆ hydrocarbyls can be optionally substituted with halo, amino, hydroxyl, C₁₋₆ alkoxy, cyano, or phosphonic acid.

In embodiments, X is NHR, NH₂, OH, or.

With respect to any relevant structural representation, such as Formula I, in embodiments, R is H or C₁₋₁₀ hydrocarbyl, including C₁₋₁₀ alkyl (e.g. methyl; C₂ alkyl, such as ethyl; C₃ alkyl, such as propyl, isopropyl, cyclopropyl, etc.; C₄ alkyl, such as linear, branched or cyclic, butyl, etc.; C₅ alkyl, C₆ alkyl, C₇ alkyl, C₈ alkyl, C₉ alkyl, or C₁₀ alkyl), C₁₋₆ alkyl, C₁₋₃ alkyl, C₁₋₁₀ alkenyl (e.g. C₂ alkenyl, such as vinyl; C₃ alkenyl, such as —CH₂—CH═CH₂; C₄ alkenyl, such as linear, branched or cyclic, butenyl, etc.; C₅ alkenyl, C₆ alkenyl, C₇ alkenyl, C₈ alkenyl, C₉ alkenyl, or C₁₀ alkenyl), C₂₋₆ alkenyl, C₂₋₄ alkenyl; optionally substituted aryl, such as phenyl; or hydrocarbyl substituted phenyl, naphthyl, etc. In embodiments, R is H, C₁₋₆ alkyl, C₁₋₃ alkyl, or CH₃.

With respect to any relevant structural representation, such as Formula I, in embodiments, A is C₁₋₁₀ hydrocarbyl, including C₁₋₁₀ alkyl (e.g. methyl; C₂ alkyl, such as ethyl; C₃ alkyl, such as propyl, isopropyl, cyclopropyl, etc.; C₄ alkyl, such as linear, branched or cyclic, butyl, etc.; C₅ alkyl, C₆ alkyl, C₇ alkyl, C₈ alkyl, C₉ alkyl, or C₁₀ alkyl), C₁₋₆ alkyl, C₁₋₃ alkyl, C₁₋₁₀ alkenyl (e.g. C₂ alkenyl, such as vinyl; C₃ alkenyl, such as —CH₂—CH═CH₂; C₄ alkenyl, such as linear, branched or cyclic, butenyl, etc.; C₅ alkenyl (such as isopentenyl), C₆ alkenyl, C₇ alkenyl, C₈ alkenyl, C₉ alkenyl, or C₁₀ alkenyl), C₂₋₆ alkenyl, C₂₋₄ alkenyl; optionally substituted aryl, such as phenyl; or hydrocarbyl substituted phenyl, naphthyl, etc.

With respect to any relevant structural representation, such as Formula I, in embodiments, A is C₃₋₆ alkyl. In embodiments, A is C₃ alkyl, such as n-propyl, isopropyl, or cyclopropyl. In embodiments, A is C₄ alkyl, such as n-butyl, t-butyl, or cyclobutyl. In embodiments, A is C₅ alky, such as n-pentyl, isopentyl, cyclopentyl, etc. In embodiments, A is C₆ alkyl, such as n-hexyl, cyclohexyl, etc. In embodiments, A is C₃₋₅ alkenyl, such as propenyl, butenyl, isopentenyl, pentenyl, etc. In embodiments, A is C₅ alkenyl, isopentenyl, or prenyl.

With respect to any relevant structural representation, such as Formula I, in embodiments, A is —(CH₂)₁₋₂—Cy, wherein Cy is optionally substituted cycloalkyl (such as cyclopropyl, cyclobutyl, cyclopentyl, cyclohexyl) or optionally substituted phenyl. In embodiments, A is

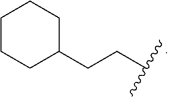 [SMILES: *CCC1CCCCC1]

In embodiments, A is

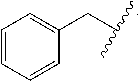 [SMILES: *Cc1ccccc1]

Examples of compounds of Formula I wherein X is NH₂ include compounds shown in Tables 1 and 2.

TABLE 1

| | |
|---|---|
| [symbol] | [symbol] |
| [symbol] | [symbol] |
| [symbol] | [symbol] |

[symbol]

indicates data missing or illegible when filed

TABLE 2

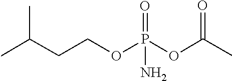

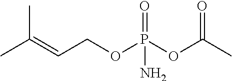

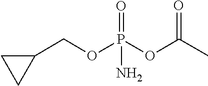

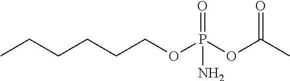

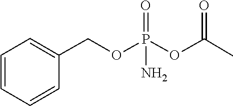

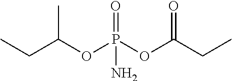

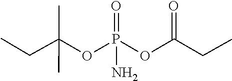

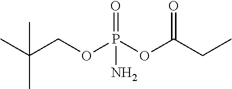

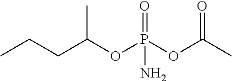

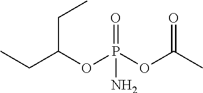

Examples of compounds of Formula I wherein X is OH include compounds shown in Table 3.

TABLE 3

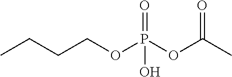

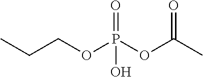

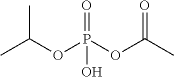

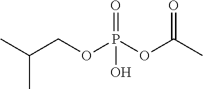

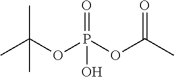

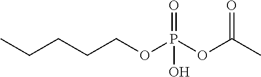

Examples of compounds of Formula I wherein X is OW, and IV is hydrocarbyl include compounds shown in Table 4.

TABLE 4

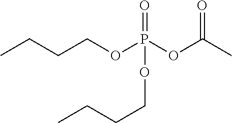 [SMILES: CCCCOP(=O)(OCCCC)OC(C)=O]

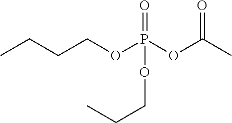

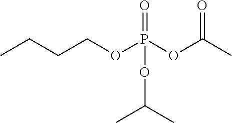

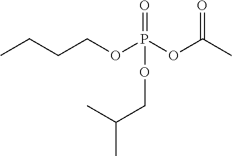 [SMILES: CCCCOP(=O)(OCC(C)C)OC(C)=O]

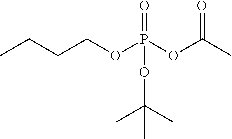 [SMILES: CCCCOP(=O)(OC(C)=O)OC(C)(C)C]

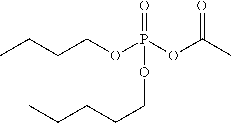 [SMILES: CCCCCOP(=O)(OCCCC)OC(C)=O]

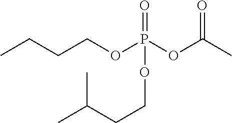 [SMILES: CCCCOP(=O)(OCCC(C)C)OC(C)=O]

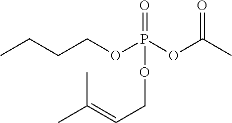 [SMILES: CCCCOP(=O)(OCC=C(C)C)OC(C)=O]

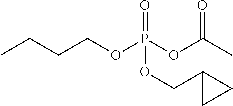 [SMILES: CCCCOP(=O)(OCC1CC1)OC(C)=O]

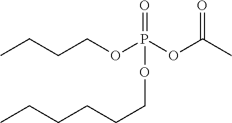 [SMILES: CCCCCCOP(=O)(OCCCC)OC(C)=O]

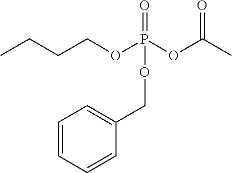 [SMILES: CCCCOP(=O)(OCc1ccccc1)OC(C)=O]

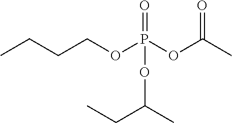 [SMILES: CCCCOP(=O)(OC(C)=O)OC(C)CC]

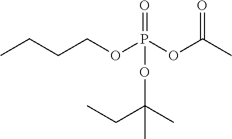 [SMILES: CCCCOP(=O)(OC(C)=O)OC(C)(C)CC]

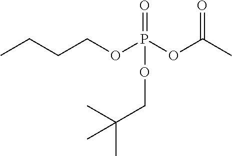 [SMILES: CCCCOP(=O)(OCC(C)(C)C)OC(C)=O]

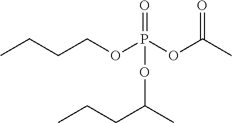 [SMILES: CCCCOP(=O)(OC(C)=O)OC(C)CCC]

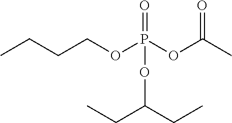 [SMILES: CCCCOP(=O)(OC(C)=O)OC(CC)CC]

In embodiments, the compounds described herein are represented by Formula II, or a salt, acid, or base thereof:

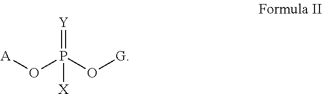

wherein A is R; Y is O or S; X is NH₂, OH, or OR; and G is R, wherein R is C₁₋₁₆ hydrocarbyl.

An example of a compound of Formula II includes a compound of Formula II wherein X is NH₂; A is (CH₂)₃CH₃; Y is O; G is OR; and R is C₁₋₁₀ hydrocarbyl.

Another example of a compound of Formula II includes a compound of Formula II wherein X is NH₂; A is (CH₂)₃CH₃; Y is O; G is OR; and R is C₅H₉, which is represented by the following structure:

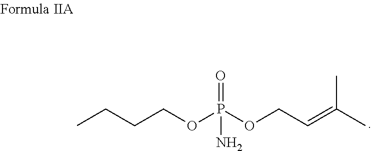

Examples of compounds of Formula II wherein X is NH₂, A is (CH₂)₃CH₃, Y is O, and G is OR, and R is C₁₋₁₀ hydrocarbyl include the compounds shown in Table 5.

TABLE 5

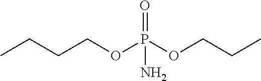

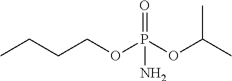

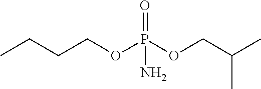

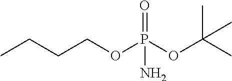

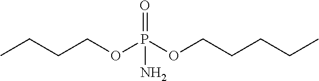

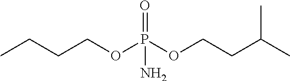

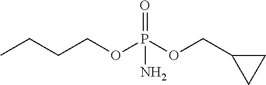

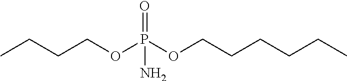

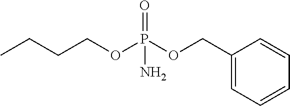 [SMILES: CCCCOP(N)(=O)OCc1ccccc1]

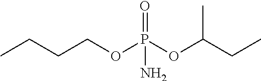

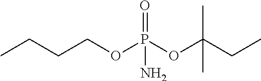

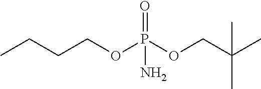

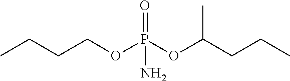

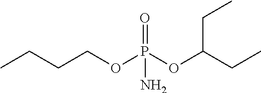

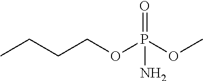

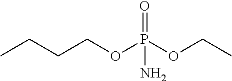

In embodiments, compounds of Formula II also include compounds of Formula II, wherein X is OH; A is R; Y is O; G is OR; and R is C₁₋₁₀ hydrocarbyl, which is represented by the following structure:

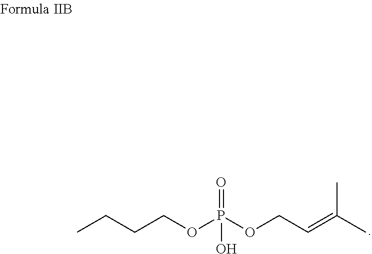

In embodiments, the compound of Formula II, wherein X is NH₂, Y is S, A is (CH₂)₃CH₃, and G is CH (CH₃)₂, has the following structure:

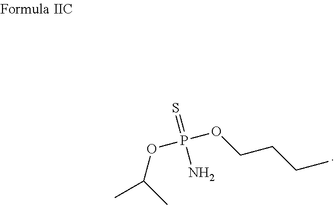

In embodiments, the compound described herein is represented by Formula III or a salt, acid, or base thereof:

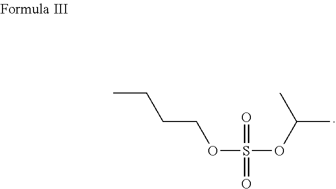

In embodiments, the compound described herein is represented by Formula IV, or a salt, acid, or base thereof:

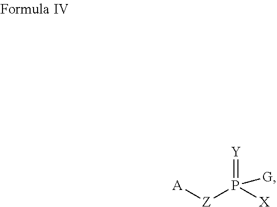

wherein A is H, C₁₋₁₆ hydrocarbyl or C₁₋₁₆ hydrocarbyl substituted with R¹; Z is O or a bond; Y is O; G is OH, H, C₁₋₆—COO-alkyl, O—C₁₋₁₆ alkyl, C₁₋₁₆ hydrocarbyl, R¹ substituted C₁₋₁₆ hydrocarbyl, CH₂NHCH₂COOH, or O-aryl; X is H, CN, —NHR, —NHOR, —NHOCOR, C₁₋₁₀ hydrocarbyl, R¹ substituted C₁₋₁₀ hydrocarbyl, or OR; and R is H, C₁₋₁₀ hydrocarbyl, or R¹ substituted C₁₋₁₀ hydrocarbyl;

R¹ is halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, or phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic acid; and

R², R³, and R⁴ are independently selected from hydrogen, C₁₋₆ hydrocarbyl, in which each of the C₁₋₆ hydrocarbyls can be optionally substituted with halo, amino, hydroxyl, C₁₋₆ alkoxy, cyano, or phosphonic acid.

In embodiments, the compound described herein is represented by Formula V, or a salt, acid, or base thereof:

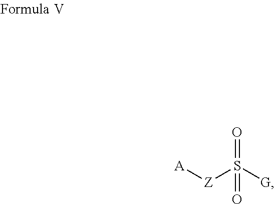

wherein A is H, C₁₋₂₀ hydrocarbyl, alkylaryl, or C₁₋₂₀ hydrocarbyl substituted with R¹; Z is O or a bond; G is OH, H, C₁₋₆—COO-alkyl, O—C₁₋₆ alkyl, C₁₋₁₆ hydrocarbyl, R¹ substituted C₁₋₁₆ hydrocarbyl, CH₂NHCH₂COOH, or O-aryl;

R¹ is halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, or phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic acid; and

R², R³, and R⁴ are independently selected from hydrogen, C₁₋₆ hydrocarbyl, in which each of the C₁₋₆ hydrocarbyls can be optionally substituted with halo, amino, hydroxyl, C₁₋₆ alkoxy, cyano, or phosphonic acid.

With respect to any relevant structural representation, such as Formula IV or Formula V, in embodiments, Z is O or a bond. In embodiments, Z is O or Z is a bond.

With respect to any relevant structural representation, such as Formula IV, in embodiments, Y is O.

With respect to any relevant structural representation, such as Formula IV or Formula V, in embodiments, G is OH, O-alkyl (such as OCH₃, OC₂H₅, OC₃H₇, OC₄H₉, OC₅H₁₁, OC₆H₁₃, etc.), C₁₋₆—COO-alkyl, C₁₋₁₆ hydrocarbyl, R¹ substituted C₁₋₁₆ hydrocarbyl, or O-aryl. In embodiments, G is OCH₃, OC₂H₅, OC₄H₉. CH₂COOCH₃, OCH₂CF₃, C₂H₄CHNH₂COOH, O-phenyl, CH₂NHCH₂COOH, or ONa.

With respect to any relevant structural representation, such as Formula IV, in embodiments, X is H, CN, NHR, NHOR, NHOCOR, C₁₋₁₀ hydrocarbyl, R¹ substituted C₁₋₁₀ hydrocarbyl, or OR; and R is H, C₁₋₁₀ hydrocarbyl, or R¹ substituted C₁₋₁₀ hydrocarbyl. In embodiments, X is H, OR, OH, OCH₃, OC₂H₅, NHR, NH₂, OCH₂CF₃, or CN.

With respect to any relevant structural representation, such as Formula IV, in embodiments, R is H or C₁₋₁₀ hydrocarbyl, including C₁₋₁₀ alkyl (e.g. methyl; C₂ alkyl, such as ethyl; C₃ alkyl, such as propyl, isopropyl, cyclopropyl, etc.; C₄ alkyl, such as linear, branched or cyclic, butyl, etc.; C₅ alkyl, C₆ alkyl, C₇ alkyl, C₈ alkyl, C₉ alkyl, or C₁₀ alkyl), C₁₋₆ alkyl, C₁₋₃ alkyl, CMO alkenyl (e.g. C₂ alkenyl, such as vinyl; C₃ alkenyl, such as CH₂—CH═CH₂, C₄ alkenyl, such as linear, branched or cyclic, butenyl, etc.; C₅ alkenyl, C₆ alkenyl, C₇ alkenyl, C₈ alkenyl, C₉ alkenyl, or C₁₀ alkenyl), C₂₋₆ alkenyl, C₂₋₄ alkenyl, or aryl or optionally substituted aryl, such as phenyl or hydrocarbyl substituted phenyl, naphthyl, etc. In embodiments, R is H, C₁₋₆ alkyl, C₁₋₃ alkyl, CH₃, C₂H₅, or CH₂CF₃.

With respect to any relevant structural representation, such as Formula IV or Formula V, in embodiments, A is H, C₁₋₂₀ hydrocarbyl, including C₁₋₁₂ alkyl (e.g. methyl; C₂ alkyl, such as ethyl; C₃ alkyl, such as propyl, isopropyl, cyclopropyl, etc.; C₄ alkyl, such as linear, branched or cyclic, butyl, etc.; C₅ alkyl, C₆ alkyl, C₇ alkyl, C₈ alkyl, C₉ alkyl, C₁₀ alkyl, C₁₁ alkyl or C₁₂ alkyl), C₁₋₆ alkyl, C₁₋₃ alkyl, C₁₋₁₀ alkenyl (e.g. C₂ alkenyl, such as vinyl; C₃ alkenyl, such as —CH₂—CH═CH₂, C₄ alkenyl, such as linear, branched or cyclic, butenyl, etc.; C₅ alkenyl (such as isopentenyl), C₆ alkenyl, C₇ alkenyl, C₈ alkenyl, C₉ alkenyl, or C₁₀ alkenyl), C₂₋₆ alkenyl, C₂₋₄ alkenyl, aryl; such as phenyl or naphthyl, alkylaryl; or optionally R¹ substituted C₁₋₂₀ hydrocarbyl.

R¹ is selected from halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, and phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic acid; and

R², R³, and R⁴ are independently selected from hydrogen, C₁₋₆ hydrocarbyl, in which each of the C₁₋₆ hydrocarbyls can be optionally substituted with halo, amino, hydroxyl, C₁₋₆ alkoxy, cyano, or phosphonic acid.

With respect to any relevant structural representation, such as Formula IV or Formula V, in embodiments, A is H. In embodiments, A is C₁₋₆ alkyl; ethyl; C₃ alkyl, such as n-propyl, isopropyl, or cyclopropyl; C₄ alkyl, such as n-butyl, t-butyl, or cyclobutyl; C₅ alky, such as n-pentyl, isopentyl, cyclopentyl, etc.; C₆ alkyl, such as n-hexyl, cyclohexyl, etc.; C₃₋₅ alkenyl, such as propenyl, butenyl, isopentenyl, pentenyl, etc.; or alkylaryl.

With respect to any relevant structural representation, such as Formula IV or Formula V, in embodiments, A is —(CH₂)₁₋₂—Cy, wherein Cy is optionally substituted cycloalkyl (such as cyclopropyl, cyclobutyl, cyclopentyl, cyclohexyl) or optionally substituted phenyl. In embodiments, A is

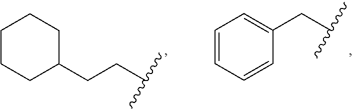 [SMILES: *CCC1CCCCC1 ; *Cc1ccccc1]

R¹ substituted alkyl, CF₃, C₂H₄OH, 1-amino-1-phenyl methyl, or 1-amino-2-phenyl-ethyl.

In embodiments of Formula IV, Y is O; Z is O; A is C₄H₉; G is OC₄H₉; and X is H, OH, OCH₃, or NH₂.

In embodiments of Formula IV, Y is O; Z is O; A is CH₂CF₃; G is CH₂COOCH₃; and X is OCH₂CF₃. In embodiments, Y is O; Z is O; A is CH₂CF₃; G is OCH₂CF₃; and X is H. embodiments, Y is O; Z is O; A is C₂H₅; G is OC₂H₅; and X is CF₂Br. In embodiments, Y is O; Z is O; A is C₂H₅; G is OC₂H₅; and X is CN.

In embodiments of Formula IV, Y is O; Z is a bond; A is C₂H₄OH; G is OCH₃; and X is OCH₃.

Examples of compounds of Formula IV include the compounds shown in Table 6.

TABLE 6

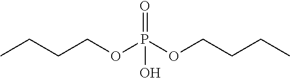

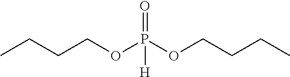

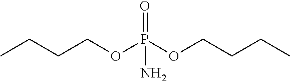

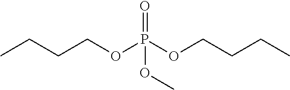

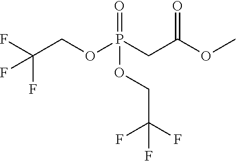 [SMILES: COC(=O)CP(=O)(OCC(F)(F)F)OCC(F)(F)F]

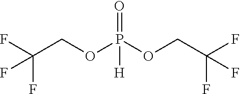 [SMILES: O=[PH](OCC(F)(F)F)OCC(F)(F)F]

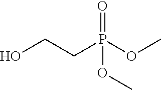

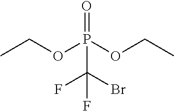

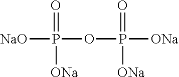

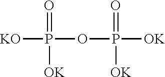

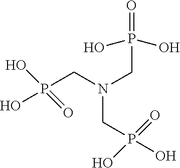 [SMILES: O=P(O)(O)CN(CP(=O)(O)O)CP(=O)(O)O]

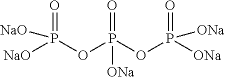

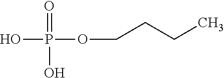

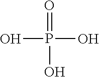

Examples of compounds of Formula IV also include compounds shown in Table 7.

TABLE 7

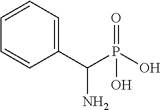 [SMILES: NC(c1ccccc1)P(=O)(O)O]

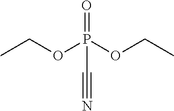

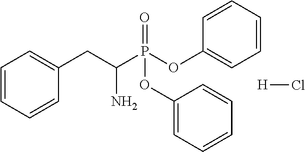

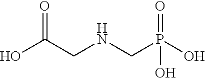

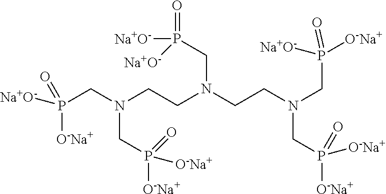

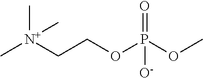

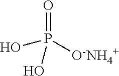

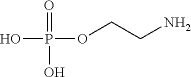

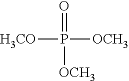

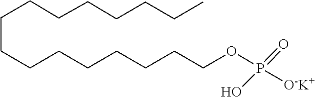

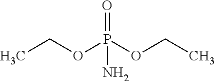

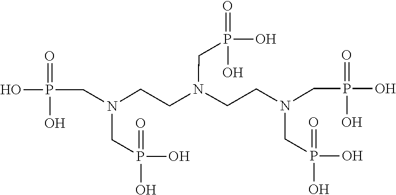 [SMILES: O=P(O)(O)CN(CCN(CP(=O)(O)O)CP(=O)(O)O)CCN(CP(=O)(O)O)CP(=O)(O)O]

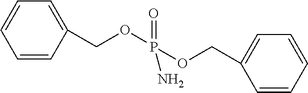 [SMILES: NP(=O)(OCc1ccccc1)OCc1ccccc1]

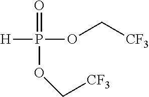 [SMILES: O=[PH](OCC(F)(F)F)OCC(F)(F)F]

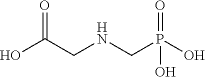

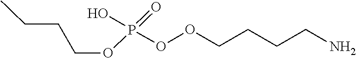

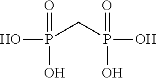

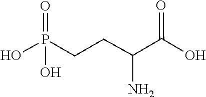 [SMILES: NC(CCP(=O)(O)O)C(=O)O]

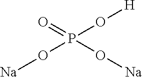

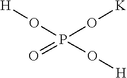

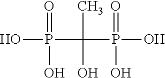

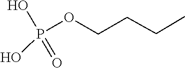

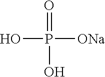

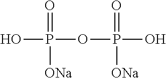

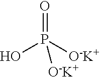

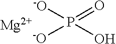

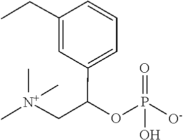 [SMILES: CCc1cccc(C(C[N+](C)(C)C)OP(=O)([O-])O)c1]

Examples of compounds of Formula IV also include the compounds shown in Table 8.

TABLE 8

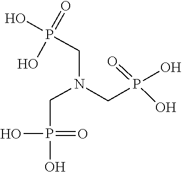 [SMILES: O=P(O)(O)CN(CP(=O)(O)O)CP(=O)(O)O]

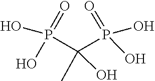

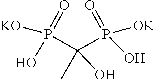

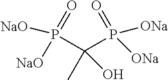

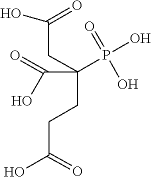 [SMILES: O=C(O)CCC(CC(=O)O)(C(=O)O)P(=O)(O)O]

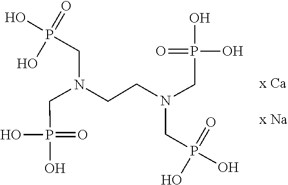 [SMILES: O=P(O)(O)CN(CCN(CP(=O)(O)O)CP(=O)(O)O)CP(=O)(O)O]

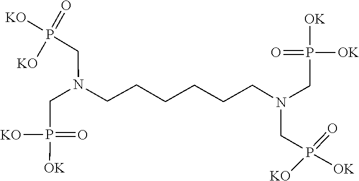

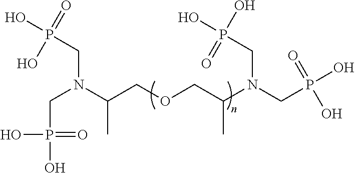

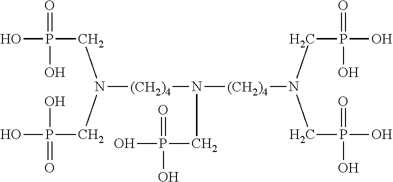

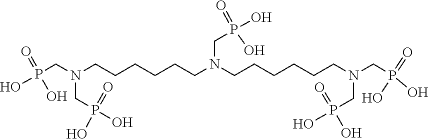 [SMILES: O=P(O)(O)CN(CCCCCCN(CP(=O)(O)O)CP(=O)(O)O)CCCCCCN(CP(=O)(O)O)CP(=O)(O)O]

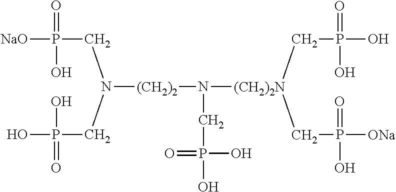

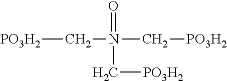

Examples of compounds of Formula IV also include compounds shown in Table 9.

TABLE 9

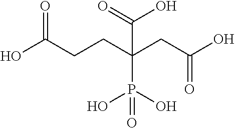 [SMILES: O=C(O)CCC(CC(=O)O)(C(=O)O)P(=O)(O)O]

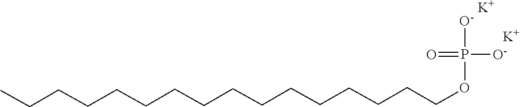 [SMILES: [K+] ; CCCCCCCCCCCCCCCCOP(=O)([O-])[O-] ; [K+]]

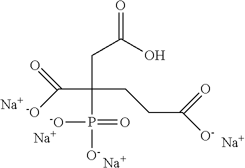

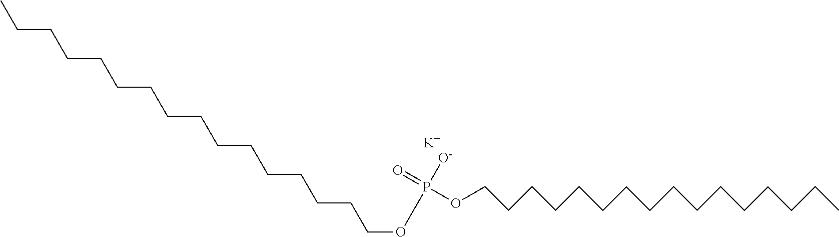 [SMILES: CCCCCCCCCCCCCCCCOP(=O)([O-])OCCCCCCCCCCCCCCCC ; [K+]]

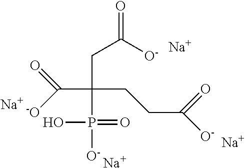

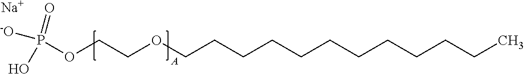

OPO₃⁻² K⁺ K⁺

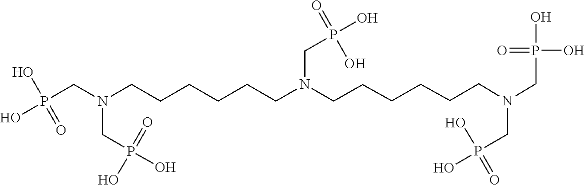 [SMILES: O=P(O)(O)CN(CCCCCCN(CP(=O)(O)O)CP(=O)(O)O)CCCCCCN(CP(=O)(O)O)CP(=O)(O)O]

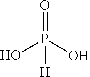

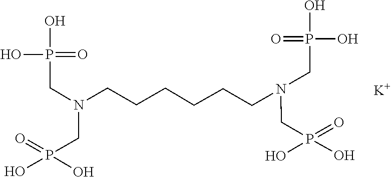 [SMILES: O=P(O)(O)CN(CCCCCCN(CP(=O)(O)O)CP(=O)(O)O)CP(=O)(O)O ; [K+]]

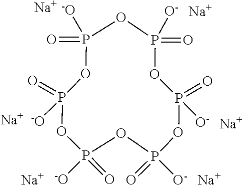

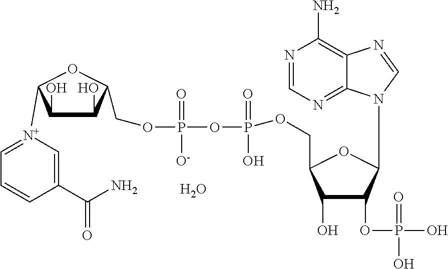

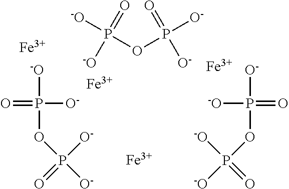

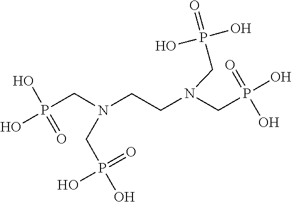 [SMILES: O=P(O)(O)CN(CCN(CP(=O)(O)O)CP(=O)(O)O)CP(=O)(O)O]

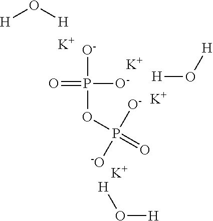

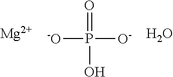

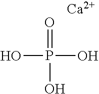

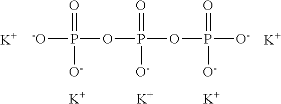

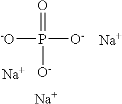

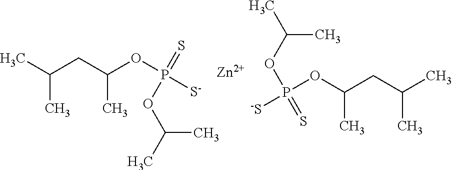

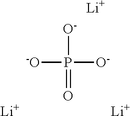

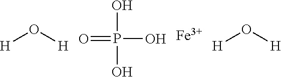

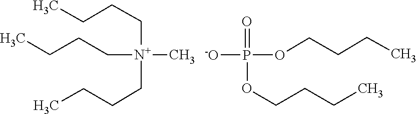

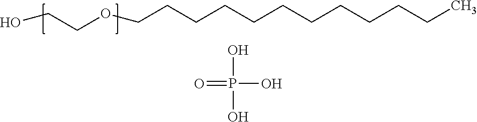

Examples of compounds of Formula V include compounds shown in Table 10.

TABLE 10

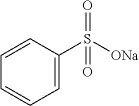

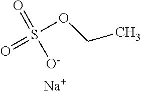

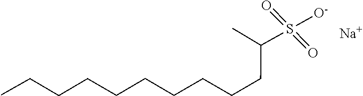 [SMILES: CCCCCCCCCCC(C)S(=O)(=O)[O-] ; [Na+]]

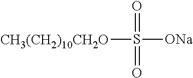

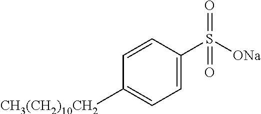

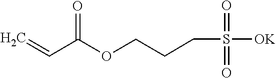

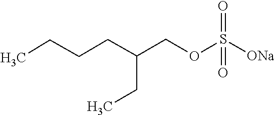

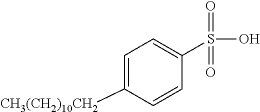

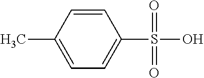

Examples of compounds of Formula V also include compounds shown in Table 11.

TABLE 11

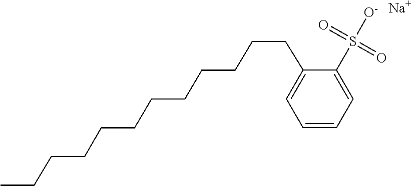 [SMILES: [Na+] ; CCCCCCCCCCCCc1ccccc1S(=O)(=O)[O-]]

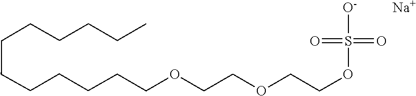 [SMILES: CCCCCCCCCCCCOCCOCCOS(=O)(=O)[O-] ; [Na+]]

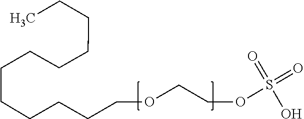

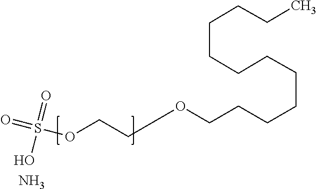

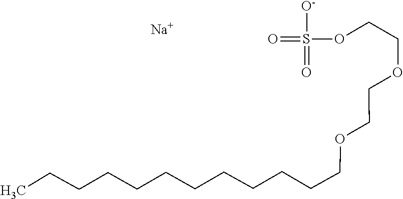 [SMILES: CCCCCCCCCCCCOCCOCCOS(=O)(=O)[O-] ; [Na+]]

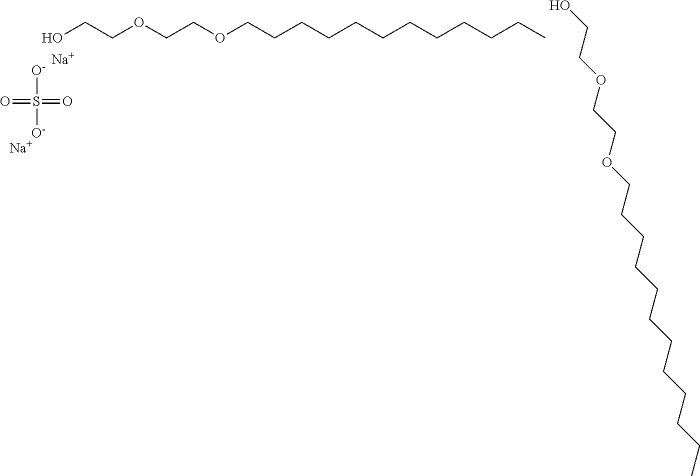

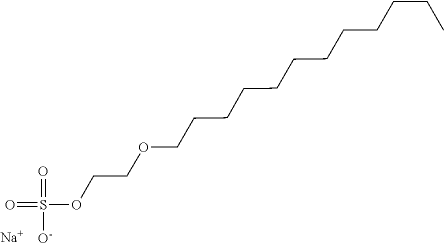 [SMILES: [Na+] ; CCCCCCCCCCCCOCCOS(=O)(=O)[O-]]

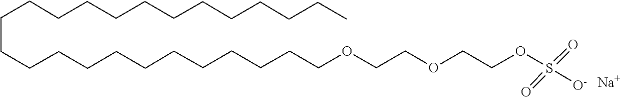 [SMILES: CCCCCCCCCCCCCCCCCCCCCCCCCOCCOCCOS(=O)(=O)[O-] ; [Na+]]

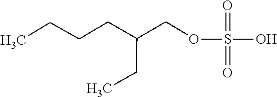

Examples of compounds of Formula V also include compounds shown in Table 12.

TABLE 12

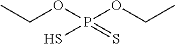

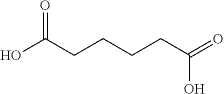

Examples of the compounds of Formulae I, II, IIA, IIB, IIC, III, IV, and V described herein also include salts, acids, or bases thereof.

In embodiments, the organophosphorous and/or organosulfurous compounds include those disclosed in U.S. Pat. No. 10,188,113 and International Application Numbers, WO 2017/151663 and WO 2018/005659, which are incorporated herein by reference in their entirety.

In embodiments, the organophosphorous and organosulfurous compounds and salts, acids, or bases thereof described herein include sodium dodecylbenzenesulfonate (SDBS), sodium dodecyl sulfate (SDS), 4-Dodecylbenzenesulfonic acid (DBSA), butyl phosphoramidate (BPA), (4-aminophenethyl)dimethylphosphine oxide (APDMPO).

In embodiments, the compounds are produced through biosynthesis. In embodiments, the compounds are produced through chemical synthesis.

In embodiments, the organophosphorous and organosulfurous compounds described herein have antimicrobial properties. As an example, they have antibacterial, antifungal, antibiofilm, antivirulent, and/or antitoxic properties. In embodiments, the antibacterial and antifungal properties including cell wall disruption, cell membrane disruption, and/or protein denaturing. In embodiments, the antivirulent properties include compounds that block virulence. In embodiments, virulence includes adhesion, invasion, colonization, ability to escape host defenses, and toxin production. In embodiments, the host defenses include innate immunity or adaptive immunity. In embodiments, the antitoxic properties include antibodies with the ability to neutralize a toxin. In embodiments, the toxin is an endotoxin or exotoxin.

The organophosphorous and organosulfurous compounds alone and in combination with one or more antimicrobial, one or more antimicrobial potentiators, and/or one or more prebiotics described herein can effectively treat persister cells. Persister cells are a subpopulation of dormant cells that form spontaneously within a biofilm which has become antimicrobial tolerant. Persister cells in their dormant state do not divide. The tolerance of antimicrobials by persister cells is different from antimicrobial resistance in that the tolerance is not inherited and is reversible.

In embodiments, the compositions described herein contain natural components including natural BPA, other organic acids, enzymes, and H₂O₂ produced by bacteria. In some embodiments, the bacteria are lactic acid bacteria. In some embodiments, the bacteria is a marine bacteria. In some embodiments, the bacteria is Aerococcus spp.

The present disclosure describes the use of the compounds described herein to treat a variety of medical diseases and disorders including microbial infections. The present disclosure describes compositions comprising one or more compounds described herein. The present disclosure also describes pharmaceutical compositions comprising one or more compounds described herein. The compositions described herein can include a carrier, and the pharmaceutical compositions described herein can include a pharmaceutically acceptable carrier.

The term “carrier” refers to a diluent, adjuvant (for example, Freund's adjuvant (complete and incomplete)), excipient, or vehicle with which the compound is administered. Pharmaceutical carriers can be sterile liquids, such as water and oils, including those of petroleum, animal, vegetable, or synthetic origins, such as peanut oil, soybean oil, mineral oil, sesame oil, and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. For the use of (further) excipients and their use see also “Handbook of Pharmaceutical Excipients”, fifth edition, R. C. Rowe, P. J. Seskey and S. C. Owen, Pharmaceutical Press, London, Chicago. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

The term “pharmaceutically acceptable” means approved by a regulatory agency of the U.S. Federal or a state government or the EMA (European Medicines Agency) or listed in the U.S. Pharmacopeia (United States Pharmacopeia—33/National Formulary—28 Reissue, published by the United States Pharmacopeial Convention, Inc., Rockville Md., publication date: April 2010) or other generally recognized pharmacopeias for use in animals, and more particularly in humans.

The one or more compounds described herein are used in combination with one or more antimicrobials, potentiator of antimicrobial, and/or probiotics.

In embodiments, the combination treatment is administered together. In embodiments, the combination treatment is administered in tandem.

The compositions described herein comprise one or more compounds described herein and one or more antimicrobials such as antibiotics from the classes of β-lactams, aminoglycosides, glycopeptides, macrolides, fluoroquinolones, sulfonamides, tetracyclines, mupirocin, salicylates, polymixins, butenafine hydrochloride, clotrimazole, miconazole nitrate, terbinafine hydrochloride, fluoroquinolone carboxylic acid derivatives, tryptophan-rich peptides, proline-rich peptides, thiosulfil, plazomicin, fosfomycin, cefepime, maxipime, pravibismane, and tolnaftate. In embodiments, the antimicrobial includes an antibiotic such as penicillin, oxacillin, gentamicin, vancomycin, piperazines, bismuth thiols, bismuth thiol complexes, bismuth dithiol complexes, chlorhexidine, itaconic acid, colistin, potassium carbonate, isothiocyanates, phenyl isothiocyanate, sodium carbonate, sodium bicarbonate, calcium phosphate, calcium carbonate, and methyl ethyl ketone.

The combination therapy described herein includes administering the composition described herein with an antibiotic and a potentiator of antibiotics (e.g. β-lactamase inhibitors), also known as adjuvants, which are molecules (with or without antibiotic activity) that enhance the activity of an existing antibiotic. Examples of potentiators of antibiotics include antibiotic adjuvant β-lactamase inhibitor, Penicillin-Binding Proteins (PBP) inhibitor, or bacterial enzyme dihydropteroate synthetase inhibitor.

β-lactamase inhibitors inhibit β-lactamases, including carbapenemases, a new group of β-lactamases. β-lactamases are enzymes produced by bacteria that provide resistance to β-lactam antibiotics such as, penicillins and cephalosporin by hydrolyzing the β-lactam ring of antibiotics, rendering them ineffective. Carbapenemases hydrolyze the carbapenem class of antimicrobials, including imipenem, biapenem, doripenem, meropenem, and ertapenem, as well as other β-lactam antibiotics.

The β-lactamase inhibitor can be a narrow-spectrum β-lactamases inhibitor. The β-lactamase inhibitor can be an extended-spectrum β-lactamases inhibitor. The β-lactamase inhibitor can be a class A, B, C, or D β-lactamase inhibitor.

The β-lactamase inhibitor can be a carbapenemase inhibitor. The carbapenemase inhibitor is a class A carbapenemase inhibitor, a class B carbapenemase inhibitor, a class C carbapenemase inhibitor, or a class D carbapenemase inhibitor.

PBP inhibitors including, boronic acid derivatives, bind to β-lactamases and/or PBPs, and inhibit them. PBPs are structurally similar to β-lactamases and confer antibiotic resistance to their host organism by acquiring mutations that allow them to continue the biosynthesis of their cell wall. In embodiments, the PBP inhibitor is a class A PBP inhibitor, a class B PBP inhibitor, a class C PBP inhibitor, or a boronic acid derivative. In embodiments, the PBP inhibitor is a high molecular mass (HMM) PBP inhibitor. In embodiments, the PBP inhibitor is a low molecular mass (LMM) PBP inhibitor.

The compositions described here further comprise a β-lactam/β-lactamase inhibitor. In embodiments, the compositions described herein further comprise a non-β-lactam/β-lactamase inhibitor.

In one embodiment, the bacterial enzyme dihydropteroate synthetase inhibitor is sulfanilamide.

The use of the compounds described herein has been found to enhance the antibiotic properties of antimicrobials, including β-lactams, aminoglycosides, glycopeptides, macrolides, fluoroquinolones, sulfonamides, tetracyclines, mupirocin, salicylates, polymixins, bacitracin, zinc, chlorhexidine, butenafine hydrochloride, clotrimazole, miconazole nitrate, terbinafine hydrochloride, fluoroquinolone carboxylic acid derivatives, tryptophan, thiosulfil, plazomicin, fosfomycin, cefepime, maxipime, pravibismane, tolnaftate, penicillin, oxacillin, gentamicin, vancomycin, piperazines, bismuth thiols, bismuth thiol complexes, bismuth dithiol complexes, chlorhexidine, itaconic acid, colistin, potassium carbonate, isothiocyanates, phenyl isothiocyanate, sodium carbonate, sodium bicarbonate, calcium phosphate, calcium carbonate, and methyl ethyl ketone or any combination thereof.

Combination therapy can result in antibiotic synergy in which two or more antimicrobials or one or more compounds described herein and one or more antimicrobials at low concentrations work together resulting in a greater potency than if each antibiotic was used separately.

The present disclosure describes combination therapy for the treatment or prevention of a disease or condition in a subject, the method comprising administration of a composition described herein to the subject. In embodiments, the composition is formulated into a pharmaceutical dosage form, a topical form, or a form for a cutaneous route of administration, including liquid spray, wash, drop, ointment, cream, gel, powder, aerosol, or the like. In embodiments, the composition is administered in a pill, tablet, capsule, lozenge, capsule, liquid, sublingual and buccal or other ingestible, transdermal, nasal, otic, ocular, vaginal, or rectal forms. In embodiments, the composition is aerosolized for delivery via the mouth, nose, or similar means for inhalation or nebulization. In embodiments, the administration is intravenous, parenteral, or any other route of administration.

Pharmaceutical compositions can be administered in a manner appropriate to the disease to be treated (or prevented). The quantity and frequency of administration will be determined by such factors as the condition of the patient, and the type and severity of the patient's disease, although appropriate dosages may be determined by clinical trials.

The components of the compositions can be supplied either separately or mixed together in unit dosage form, for example, as a dry formulation for dissolution such as a lyophilized powder, freeze-dried powder, or water-free concentrate in a hermetically sealed container such as an ampoule or sachet. The components of the compositions can also be supplied as admixed liquid formulation (i.e. injection or infusion solution) in a hermetically sealed container such as an ampoule, sachet, a pre-filled syringe, or autoinjector, or a cartridge for a reusable syringe or applicator (e.g. pen or autoinjector). Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In a combination therapy, the one or more additional agents can be a separate composition and administered to the subject separately from the organophosphorous and/or organosulfurous composition. The one or more additional agents can be administered sequentially or simultaneously with the organophosphorous and/or organosulfurous composition.

The present disclosure also describes a method of treating or preventing an infection, disease, or condition in a subject by administration of an effective amount of the compositions described herein to a subject. The subject is a human, an animal, and/or other living organisms. In embodiments, the disease or condition is a single microbial infection or a polymicrobial infection (including but not limited to bacterial, viral, or fungal infections).

In embodiments, the microorganism is sensitive to antimicrobials or is antimicrobial-resistant (AMR). In embodiments, the microbe is sensitive or resistant to one or more antimicrobials including, β-lactams, aminoglycosides, glycopeptides, macrolide, fluoroquinolones, methicillin, vancomycin, carbapenem, clarithromycin, cephalosporin, ampicillin, penicillin, erythromycin, streptomycin, tetracycline, amoxicillin, ciprofloxacin, fluoroquinolone, sulfonamides, tetracyclines, mupirocin, salicylates, polymixins, cefepime, maxipime, pravibismane, silver, salicylic acid, neomycin sulfate, polymyxins, bacitracin, zinc, alcohol, or chlorhexidine.

As used herein, the term “antimicrobial” refers to a substance that destroys or inhibits the growth of microorganisms. In embodiments, the fungal, bacterial, or viral infection being treated is pathogenic to humans, animals, and/or other living organisms. In embodiments, the fungal infection is resistant to one or more antimicrobials including, fluconazole, amphotericin, echinocandins, itraconazole, flucytosine, butenafine hydrochloride, clotrimazole, miconazole nitrate, terbinafine hydrochloride, and tolnaftate.

The infection can be caused by a microorganism or a combination of microorganisms. Examples of microorganisms include Staphylococcus aureus, Staphylococcus epidermidis, Acinetobacter baumannii, Pseudomonas aeruginosa, Enterobacteriaceae, Escherichia coli, Salmonella enterica, Salmonella bongori, Enterococcus faecium, Helicobacter pylori, Campylobacter spp., Streptococcus pneumoniae, Streptococcus mutans, Streptococcus gordonii, Streptococcus pyogenes, Haemophilus influenzae, Shigella spp., Klebsiella pneumoniae, Clostridium difficile, Bacillus anthracis, Yersinia pestis, Francisella tularensis, Corynebacterium spp., Micrococcus luteus, Micrococcus lylae, Micrococcus roseus, Cutibaceterium acnes, Vibrio vulnificus, Vibrio cholerae, Vibrio parahaemolyticus, Propionibacterium acnes, Neisseria gonorrhoeae, Burkholderia cepacia, Burkholderia mallei, Burkholderia pseudomallei, Ralstonia pickettii, Cuprividus metallidurans, and other biofilm-forming microorganisms, including bacteria and/or viruses.

In embodiments, the infection is caused by a species of fungi, including Candida albicans, Candida auris, Aspergillus fumigatus, Aspergillus flavus, Apohysomyces sp., Blastomyces dermatitides, Coccidioides posadasii, Cryptococcus neoformans, Fusarium spp., Histoplasma capsulatum, Pneumocystis jirovecii, Rhizopus oryzae, Scedosporium spp., and/or other biofilm-forming fungi.

The term “effective amount” refers to an amount, concentration, or dosage sufficient to produce a desired result. The effective amount may vary depending on one or more compounds described herein and additional agents that are being used, and may also depend on a variety of factors and conditions related to the patient being treated and the severity of the disorder. For example, if the composition is to be administered in vivo, factors to be considered include the age, weight, and health of the patient as well as dose-response curves and toxicity data obtained in preclinical animal trials. The determination of an effective amount is within the ability of those skilled in the art. In embodiments, the compounds described herein are administered at a concentration of 0.1 μg/ml to 300 μg/ml. In embodiments, the compounds described herein is administered at a concentration of 0.1 μg/ml, 0.5 μg/ml, 1.0 μg/ml, 2.5 μg/ml, 5μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, or 100 μg/ml. In embodiments, the antimicrobial is administered at a concentration of 0.01 μg/ml to 30 μg/ml. In embodiments, the antimicrobial is administered at a concentration of 0.01 μg/ml, 0.1 μg/ml, 1.0 μg/ml, 2.5 μg/ml, 5 μg/ml, 10 μg/ml, 15 μg/ml, 20 μg/ml, 25 μg/ml, or 30 μg/ml.

The compounds described herein are used in a solution buffered to a pH 4-5. In embodiments, the compounds described herein are anionic, cationic, or nonionic.

The composition can include one or more additional agents, for example, one or more antimicrobials, one or more antimicrobial potentiators, and/or one or more probiotics.

The composition described herein can also include a non-steroidal anti-inflammatory drug (NSAID). Examples of NSAIDs include aspirin, ibuprofen, and naproxen.

The composition described herein includes one or more antihistamine. In embodiments, the antihistamine drug is an H₁-antihistamines, H₂-antihistamines, H₃-antihistamines, and H₄-antihistamines.

The composition described herein includes one or more probiotics such as Bifidobacteria or Lactobacillus. Examples of specific strains of Bifidobacteria include B. animalis, B. breve, B. lactis, or B. longum. Examples of specific strains of Lactobacillus include L. acidophilus or L. reuteri.

The present disclosure also describes a method of preventing the growth of or removing or killing one or more microorganisms from a clinical surface, the method including contacting the clinical surface with a composition including one or more organophosphorous or organosulfurous compounds and one or more antimicrobials, antimicrobial potentiators, and/or prebiotics. The clinical surface could be a surface in a hospital, a body surface of a subject, or tissue or organ surface of a subject. The composition can be formulated as one or more of a cleaning agent, dispersant, surfactant, antiseptic, anti-odor agent, anti-biofilm agent (including but not limited to biofilm removal agent, biofilm disruption agent, biofilm inhibition agent, etc.), antifouling agent (including but not limited to fouling removal agent, fouling disruption agent, fouling inhibition agent) or antimicrobial growth agent (including but not limited to disrupt, remove, prevent, and/or inhibit microbial growth and to kill microorganisms, including bacteria, fungi, viruses, and other microorganisms). The composition can be formulated as a therapeutic agent or an antibiotic agent for cleaning or treating the body surface or tissue or organ surface of a subject. The surfaces can be inert surfaces, nano surfaces, or organic surfaces.

The composition described herein can be formulated on a medical device, including in a coating, paint, chemically bound material, or the like. In one embodiment, the composition is formulated in a liquid bandage. In embodiments, the composition is formulated in a hand sanitizer. In embodiments, the composition described herein is formulated with nanotechnology. In embodiments, the composition described herein is formulated with controlled-release technology. In embodiments, the composition described herein comprises a polymeric material. In embodiments, the composition comprises a polymeric material and/or substitutes for polymeric material, such as polymers produced from algae. In embodiments, the composition described herein comprises natural and synthetic materials, including cotton.

The present disclosure also describes a method of preventing or treating microbes on or in a medical device, the method comprising contacting a medical device with an effective amount of a composition comprising one or more of compounds described herein, and one or more antimicrobials, one or more antimicrobial potentiators, and one or more prebiotics.

Methods disclosed herein include treating, preventing, or mitigating microbial infections of or alleviating the symptoms of microbial infections of subjects. Subjects include humans; veterinary animals, such as dogs, cats, reptiles, birds, and the like; livestock such as horses, cattle, goats, pigs, chickens, and other mammals; and research animals, such as monkeys, rats, mice, fish, and other mammals. Subjects in need of a treatment (in need thereof) are subjects having or at risk of developing microbial infections.

The terms “prevent” or “prevention” refers to the prevention of the onset, recurrence, or spread of a microbial infection or one or more symptoms of a microbial infection. The term includes administration of a compound described herein before the onset of symptoms in particular to subjects at risk of developing a microbial infection. The term includes the inhibition or reduction of one or more symptoms associated with the microbial infection. The term “prevention” can be used interchangeably with the term “prophylactic treatment”.

As will be understood by one of ordinary skill in the art, each embodiment disclosed herein can comprise, consist essentially of or consist of its particular stated element, step, ingredient, or component. Thus, the terms “include” or “including” should be interpreted to recite: “comprise, consist of, or consist essentially of” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. In embodiments, lack of a material effect is evidenced by lack of a statistically significant reduction in the embodiment's ability to perform a function in vitro or in vivo, for example, killing microorganisms, such as viruses, bacteria, or fungi.

All numbers expressing quantities of ingredients, properties such as concentration, effective concentration, dosage, percentage, frequency, instructions, directions, so forth used in the specification and claims are to be understood as being modified in all instances by the term “about,” unless indicated to the contrary. Accordingly, the numerical parameters set forth in the specification and attached claims are approximations that may vary depending upon the desired properties sought to be obtained. At the very least, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±15% of the stated value; ±10% of the stated value; ±5% of the stated value; ±4% of the stated value; ±3% of the stated value; ±2% of the stated value; ±1% of the stated value; or ±any percentage between 1% and 20% of the stated value.

Recitation of ranges of values herein is merely intended to serve as a shorthand method of referring individually to each separate value falling within the range. Unless otherwise indicated herein, each individual value is incorporated into the specification as if it were individually recited herein. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.5, 2.7, 3, 4, 5, 5.1, 5.3, 5.8, and 6. This applies regardless of the breadth of the range. Moreover, any ranges cited herein are inclusive of the upper and lower limit of the ranges.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The following exemplary embodiments and examples illustrate exemplary methods provided herein. These exemplary embodiments and examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXEMPLARY EMBODIMENTS

The following are exemplary embodiments:
1. A method of treating or preventing a disease or condition caused by one or more microorganisms in a subject, the method comprising administrating an effective amount of a composition comprising one or more of organophosphorous or organosulfurous compounds, or salts, acids, or bases thereof.
2. The method of embodiment 1, wherein the one or more of organophosphorous or organosulfurous compounds, or salts, acids, or bases thereof have the structure of Formula I, Formula II, Formula III, Formula IV, or Formula V.
3. The method of embodiment 1 or 2, wherein the one or more oganophosphorous or organosulfurous compounds, or salts, acids, or bases thereof are shown in Table 1, 2, 3, 4, 5, 6, 7, 8, 9, 10, 11, or 12, or have the structure of Formula IIA, Formula IIB, or Formula IIC.
4. The method of any one of embodiments 1-3, wherein the composition further includes one or more antimicrobials, antimicrobial potentiators, or prebiotics.
5. The method of any one of embodiments 1-4, wherein the composition is administered topically, orally, intravenously, or aerosolized.
6. The method of any one of embodiments 1-5, wherein the disease or condition is pathogenic to humans, animals, other living organisms, or a combination thereof.
7. The method of any one of embodiments 1-6, wherein the one or more microorganisms are bacteria, fungi, viruses, or a combination thereof.
8. The method of any one of embodiments 1-7, wherein the disease or condition is caused by Staphylococcus aureus, Staphylococcus epidermidis, Acinetobacter baumannii, Pseudomonas aeruginosa, Enterobacteriaceae, Escherichia coli, Salmonella enterica, Salmonella bongori, Enterococcus faecium, Helicobacter pylori, Campylobacter spp., Neisseria gonorrhoeae, Streptococcus pneumoniae, Streptococcus mutans, Streptococcus gordonii, Streptococcus pyogenes, Haemophilus influenzae, Shigella spp., Klebsiella pneumoniae, Clostridium difficile, Bacillus anthracis, Yersinia pestis, Francisella tularensis, Burkholderia mallei, Burkholderia pseudomallei, Corynebacterium spp., Micrococcus luteus, Micrococcus lylae, Micrococcus roseus, Cutibaceterium acnes, Vibrio vulnificus, Vibrio cholerae, Vibrio parahaemolyticus, Propionibacterium acnes, Neisseria gonorrhoeae, Burkholderia cepacia, Burkholderia mallei, Burkholderia pseudomallei, Ralstonia pickettii, Cuprividus metallidurans, or a combination thereof.
9. The method of any one of embodiments 1-7, wherein the disease or condition is caused by Candida albicans, Candida auris, Aspergillus fumigatus, Aspergillus flavus, Apohysomyces sp., Blastomyces dermatitides, Coccidioides posadasii, Cryptococcus neoformans, Fusarium spp., Histoplasma capsulatum, Pneumocystis jirovecii, Rhizopus oryzae, Scedosporium spp., or a combination thereof.
10. The method of any one of embodiments 1-9, wherein the one or more microorganisms are methicillin-sensitive, methicillin-resistant, vancomycin-intermediate, vancomycin-resistant, carbapenem-resistant, clarithromycin-resistant, cephalosporin-resistant, ampicillin-resistant, penicillin-non-susceptible, fluconazole-resistant, amphotericin-resistant, echinocandins-resistant, mupirocin-resistant, erythromycin-resistant, streptomycin-resistant, tetracycline-resistant, amoxicillin-resistant, ciprofloxacin-resistant, silver-resistant, salicylic acid-resistant, neomycin sulfate-resistant, polymyxin B sulfate-resistant, bacitracin-resistant, zinc-resistant, alcohol-tolerant, alcohol-resistant, chlorhexidine-resistant, fluoroquinolone-resistant, or a combination thereof.
11. The method of any one of embodiments 1-10, wherein the one or more organophosphorous or organosulfurous compounds, or salts, acids, or bases thereof include sodium dodecylbenzenesulfonate (SDBS), sodium dodecyl sulfate (SDS), 4-Dodecylbenzenesulfonic acid (DBSA), butyl phosphoramidate (BPA), (4-aminophenethyl)dimethylphosphine oxide (APDMPO), or a combination thereof.
12. The method of any one of embodiments 1-11, wherein the one or more organophosphorous or organosulfurous compounds, or salts, acids or bases thereof are each present in the composition at a concentration of 0.1 μg/ml to 300 μg/ml.
13. The method of any one of embodiments 1-12, wherein the one or more organophosphorous or oganosulfurous compounds, or salts, acids, or bases thereof are in the form of nanoparticles.
14. The method of any one of embodiments 1-13, wherein the composition further includes one or more antimicrobials.
15. The method of embodiment 14, wherein the one or more antimicrobials include antibiotic, antiseptic, β-lactam, aminoglycoside, glycopeptide, macrolide, fluoroquinolone, sulfonamide, tetracycline, mupirocin, salicylate, polymixin, butenafine hydrochloride, clotrimazole, miconazole nitrate, terbinafine hydrochloride, fluoroquinolone carboxylic acid derivatives, tryptophan, thiosulfil, plazomicin, fosfomycin, cefepime, maxipime, pravibismane, tolnaftate, or a combination thereof.
16. The method of embodiment 14 or 15, wherein the one or more antimicrobials include piperazines, bismuth thiols, bismuth thiol complexes, bismuth dithiol complexes, chlorhexidine, itaconic acid, colistin, potassium carbonate, isothiocyanates, phenyl isothiocyanate, sodium carbonate, sodium bicarbonate, calcium phosphate, calcium carbonate, methyl ethyl ketone, or a combination thereof.
17. The method of embodiment 14 or 15, wherein the one or more antimicrobials include iodine, povidone-iodine, acetic acid, hydrogen peroxide, peroxide, peracetic acid, sodium hypochlorite, or a combination thereof.
18. The method of any one of embodiments 4-17, wherein the one or more antimicrobials, antimicrobial potentiators, or prebiotics are in the form of nanoparticles.
19. The method of any one of embodiments 1-20. The method of any one of embodiments 1-19, wherein the composition further includes one or more silver compounds.
21. The method of embodiment 20, wherein the one or more silver compounds include a silver ion, silver particle, silver nanoparticle, metallic silver, colloidal silver, silver chloride, or a combination thereof.
22. The method of embodiment 20 or 21, wherein the concentration of each of the one or more silver compounds is 0.01 μg/ml to 30 μg/ml.
23. The method of any one of embodiments 1-22, wherein the composition further includes one or more antibiotic adjuvants.
24. The method of embodiment 23, wherein the one or more antibiotic adjuvants include a β-lactamase inhibitor, penicillin-binding protein inhibitor, dihydropteroate synthetase inhibitor, iron chelator, or a combination thereof.
25. The method of embodiment 23 or 24, wherein the concentration of each of the one or more antibiotic adjuvants is 0.01 μg/ml to 500 μg/ml.
26. The method of any one of embodiments 1-25, wherein the composition further includes one or more non-steroidal anti-inflammatory drugs.
27. The method of embodiment 26, wherein the one or more non-steroidal anti-inflammatory drugs include aspirin.
28. The method of embodiment 26 or 27, wherein the concentration of each of the one or more non-steroidal anti-inflammatory drugs is 0.01 μg/ml to 50 μg/ml.
29. The method of any one of embodiments 1-28, wherein the composition further includes one or more antibiofilm agents.
30. The method of embodiment 29, wherein the one or more antibiofilm agents interfere with bacterial signals, interfere with bacterial systems, interfere with the physical attachment, induce bacterial cells to detach or interfere with a combination thereof.
31. The method of embodiment 29 or 30, wherein the one or more antibiofilm agents include a protein kinase inhibitor, a quorum sensing signal inhibitor, a compound that induces the bacterial cells to detach, or a combination thereof.
32. The method of embodiment 31, wherein the protein kinase inhibitor includes a serine/threonine kinase inhibitor, tyrosine kinase inhibitor, or serine/threonine/tyrosine kinase inhibitor.
33. The method of embodiment 31, wherein the quorum sensing signal inhibitor includes Avellanin C, p-nitrophenyl glycerol, tannic acid, or RNAIII inhibiting peptide.
34. The method of any one of embodiments 31-33, wherein the concentration of the protein kinase inhibitor or quorum sensing signal inhibitor is 0.01 μg/ml to 500 μg/ml.
35. The method of any one of embodiments 29-34, wherein the one or more antibiofilm agents that interferes with physical attachment of the cells to surfaces include compounds that interfere with adhesins, pili, extracellular polymeric matrices, or a combination thereof.
36. The method of any one of embodiments 29-35, wherein the one or more antibiofilm agents that induce the bacterial cells to detach include sugars, amino acids, carbon sources, salt, other nutrients, or a combination thereof.
37. The method of any one of embodiments 29-36, wherein the one or more antibiofilm agents are in the form of nanoparticles.
38. The method of any one of embodiments 1-37, wherein the composition further includes a microbially produced metabolite, enzyme, bacteriocin, and/or acid.
39. The method of embodiment 38, wherein the microbially produced metabolite includes propionate, butyrate, acetate, serine endopeptidase, succinic acid, lactic acid, formic acid, propionic acid, nisin, actagardine, durancin 61A, and/or PsVP-10.
40. The method of embodiment 38 or 39, wherein the concentration of each of the microbially produced metabolite, enzyme, bacteriocin, or acid is 0.01 μg/ml to 500 μg/ml and/or 1% (v/v) to 10% (v/v).
41. A method of preventing growth of or removing or killing one or more microorganisms from a clinical surface, the method comprising contacting the clinical surface with a composition comprising an effective amount of a composition comprising one or more of organophosphorous or organosulfurous compounds, or salts, acids or bases thereof, and one or more of antimicrobials, antimicrobial potentiators, probiotics, or prebiotics.
42. The method of embodiment 41, wherein the one or more of organophosphorous or organosulfurous compounds, or salts, acids, or bases thereof have the structure of Formula I, Formula II, Formula III, Formula IV, or Formula V.
43. The method of embodiment 41 or 42, wherein the one or more oganophosphorous or organosulfurous compounds, or salts, acids, or bases thereof have the structure shown in Table 1, 2, 3, 4, 5, 6, 7, 8, 9, 10, 11, or 12 or have the structure of Formula IIA, Formula IIB, or Formula IIC.
44. The method of any one of embodiments 41-43, wherein the composition includes an antiseptic, a cleaning agent, a dispersant, a surfactant, an anti-odor agent, an anti-biofilm agent, an anti-fouling agent, an antimicrobial, a therapeutic agent, a prophylactic antibiotic, or a combination thereof.
45. The method of any one of embodiments 41-44, wherein the one or more microorganisms are pathogenic to humans, animals, other living organisms, or a combination thereof.
46. The method of any one of embodiments 41-44, wherein the one or more microorganisms include bacteria, fungi, viruses, or a combination thereof.
47. The method of any one of embodiments 41-46, wherein the one or more microorganisms include Staphylococcus aureus, Staphylococcus epidermidis, Acinetobacter baumannii, Pseudomonas aeruginosa, Enterobacteriaceae, Escherichia coli, Salmonella enterica, Salmonella bongori, Enterococcus faecium, Helicobacter pylori, Campylobacter spp., Neisseria gonorrhoeae, Streptococcus pneumoniae, Streptococcus mutans, Streptococcus gordonii, Streptococcus pyogenes, Haemophilus influenzae, Shigella spp., Klebsiella pneumoniae, Clostridium difficile, Bacillus anthracis, Yersinia pestis, Francisella tularensis, Burkholderia mallei, Burkholderia pseudomallei, Corynebacterium spp., Micrococcus luteus, Micrococcus lylae, Micrococcus roseus, Cutibaceterium acnes, Vibrio vulnificus, Vibrio cholerae, Vibrio parahaemolyticus, Propionibacterium acnes, Neisseria gonorrhoeae, Burkholderia cepacia, Burkholderia mallei, Burkholderia pseudomallei, Ralstonia pickettii, Cuprividus metallidurans, or a combination thereof.
48. The method of any one of embodiments 41-46, wherein the one or more microorganisms include Candida albicans, Candida auris, Aspergillus fumigatus, Aspergillus flavus, Apohysomyces sp., Blastomyces dermatitides, Coccidioides posadasii, Cryptococcus neoformans, Fusarium spp., Histoplasma capsulatum, Pneumocystis jirovecii, or Rhizopus oryzae, Scedosporium spp., or a combination thereof.
49. The method of any one of embodiments 41-48, wherein the one or more microorganisms are methicillin-sensitive, methicillin-resistant, vancomycin-intermediate, vancomycin-resistant, carbapenem-resistant, clarithromycin-resistant, cephalosporin-resistant, ampicillin-resistant, penicillin-non-susceptible, fluconazole-resistant, amphotericin-resistant, echinocandins-resistant, mupirocin-resistant, erythromycin-resistant, streptomycin-resistant, tetracycline-resistant, amoxicillin-resistant, ciprofloxacin-resistant, silver-resistant, salicylic acid-resistant, neomycin sulfate-resistant, polymyxin B sulfate-resistant, bacitracin-resistant, zinc-resistant, alcohol-tolerant, alcohol-resistant, chlorhexidine-resistant, fluoroquinolone-resistant, or a combination thereof.
50. The method of any one of embodiments 41-49, wherein the one or more organophosphorous or organosulfurous compounds, or salts, acids, or bases thereof include sodium dodecylbenzenesulfonate (SDBS), sodium dodecyl sulfate (SDS), 4-Dodecylbenzenesulfonic acid (DBSA), butyl phosphoramidate (BPA), (4-aminophenethyl)dimethylphosphine oxide (APDMPO), or a combination thereof.
51. The method of any one of embodiments 41-50, wherein the one or more organophosphorous or organosulfurous compounds, or salts, acids or bases thereof are each present in the composition at a concentration of 0.1 μg/ml to 300 μg/ml.
52. The method of any one of embodiments 41-51, wherein the one or more antimicrobials include an antibiotic, an antiseptic, a β-lactam, aminoglycoside, glycopeptide, macrolide, fluoroquinolone, sulfonamide, tetracycline, mupirocin, salicylate, polymixin, butenafine hydrochloride, clotrimazole, miconazole nitrate, terbinafine hydrochloride, fluoroquinolone carboxylic acid derivatives, tryptophan, thiosulfil, plazomicin, fosfomycin, tolnaftate, cefepime, maxipime, pravibismane, or a combination thereof.
53. The method of any one of embodiments 41-52, wherein the one or more antimicrobials include piperazines, bismuth thiols, bismuth thiol complexes, bismuth dithiol complexes, chlorhexidine, itaconic acid, colistin, potassium carbonate, isothiocyanates, phenyl isothiocyanate, sodium carbonate, sodium bicarbonate, calcium phosphate, calcium carbonate, methyl ethyl ketone, or a combination thereof.
54. The method of any one of embodiments 41-52, wherein the one or more antimicrobials include iodine, povidone-iodine, acetic acid, hydrogen peroxide, peroxide, peracetic acid, sodium hypochlorite, or a combination thereof.
55. The method of any one of embodiments 41-52, wherein the concentration of each of the one or more antimicrobials is 0.01 μg/ml to 30 μg/ml.
56. The method of any one of embodiments 41-55, wherein the composition further includes one or more silver compounds.
57. The method of embodiment 56, wherein the one or more silver compounds include a silver ion, silver particle, silver nanoparticle, metallic silver, colloidal silver, silver chloride, or a combination thereof.
58. The method of embodiment 56 or 57, wherein the concentration of each of the one or more silver compounds is 0.01 μg/ml to 30 μg/ml.
59. The method of any one of embodiments 41-58, wherein the composition includes one or more antibiotic adjuvants.
60. The method of embodiment 59, wherein the one or more antibiotic adjuvants include a β-lactamase inhibitor, penicillin-binding protein inhibitor, dihydropteroate synthetase inhibitor, iron chelator, or a combination thereof.
61. The method of embodiment 59 or 60, wherein the concentration of each of the one or more antibiotic adjuvants is 0.01 μg/ml to 500 μg/ml.
62. The method of any one of embodiments 41-61, wherein the composition further includes one or more non-steroidal anti-inflammatory drugs.
63. The method of embodiment 62, wherein the non-steroidal anti-inflammatory drug includes aspirin.
64. The method of embodiment 62 or 63, wherein the concentration of each of the one or more non-steroidal anti-inflammatory drugs is 0.01 μg/ml to 50 μg/ml.
65. The method of any one of embodiments 41-64, wherein the composition further includes one or more antibiofilm agents.
66. The method of embodiment 65, wherein the one or more antibiofilm agents interfere with bacterial signals, interfere with bacterial systems, interfere with the physical attachment, induce bacterial cells to detach or interfere a combination thereof.
67. The method of embodiment 65 or 66, wherein the one or more antibiofilm agents include a protein kinase inhibitor, a quorum sensing signal inhibitor, a compound that induces the bacterial cells to detach, or a combination thereof.
68. The method of embodiment 67, wherein the protein kinase inhibitor includes a serine/threonine kinase inhibitor, tyrosine kinase inhibitor, or serine/threonine/tyrosine kinase inhibitor.
69. The method of embodiment 67, wherein the quorum sensing signal inhibitor includes Avellanin C, p-nitrophenyl glycerol, tannic acid, or RNAIII inhibiting peptide.
70. The method of any one of embodiments 67-69, wherein the concentration of the protein kinase inhibitor or quorum sensing signal inhibitor is 0.01 μg/ml to 500 μg/ml.
71. The method of any one of embodiments 65-70, wherein the one or more antibiofilm agents that interferes with physical attachment of the cells to surfaces include compounds that interfere with adhesins, pili, extracellular polymeric matrices, or a combination thereof.
72. The method of any one of embodiments 65-71, wherein the one or more antibiofilm agents that induce the bacterial cells to detach include sugars, amino acids, carbon sources, salt, other nutrients, or a combination thereof.
73. The method of any one of embodiments 65-72, wherein the one or more antibiofilm agents are in the form of nanoparticles.
74. The method of any one of embodiments 41-73, wherein the composition further includes a microbially produced metabolite, enzyme, bacteriocin, and/or acid.
75. The method of embodiment 74, wherein the microbially produced metabolite includes propionate, butyrate, acetate, serine endopeptidase, succinic acid, lactic acid, formic acid, propionic acid, nisin, actagardine, durancin 61A, and/or PsVP-10.
76. The method of embodiment 74 or 75, wherein the concentration of each of the microbially produced metabolite, enzyme, bacteriocin, or acid is 0.01 μg/ml to 500 μg/ml and/or 1% (v/v) to 10% (v/v).
77. The method of any one of embodiments 41-76, wherein the one or more organophosphorous or oganosulfurous compounds, or salts, acids, or bases thereof are in the form of nanoparticles.
78. The method of any one of embodiments 41-77, wherein the one or more antimicrobials, antimicrobial potentiators, or prebiotics are in the form of a nanoparticle.

### EXAMPLES

The following Examples are illustrative of specific embodiments of the disclosure, and various uses thereof. They are set forth for explanatory illustration only and are not to be construed as limiting the disclosure in any way.

### Materials Used in Examples

Chemicals and Media. The effects of the organophosphorous and/or organosulfurous compounds alone and in combination with existing antibiotics were studied. BPA (98%) and APDMPO (90.2%) were obtained from Syngene International (Bangalore, India). SDBS (>90%), SDS (98.5%), DBSA (95%), and methicillin (>85%), gentamicin (>98%), penicillin G (>95%), vancomycin (95%), streptomycin sulfate salt (95%), tetracycline hydrochloride (95%), ampicillin sodium salt (95%), amoxicillin (<=100%), and ciprofloxacin (>98%) were obtained from Sigma-Aldrich (St. Louis, MO). All culture media (BD BBL) was prepared as per the manufacturer's instructions.

Microorganisms. Five bacterial species were obtained from the American Type Culture Collection (ATCC) (ATCC; Manassas, VA) including, Staphylococcus aureus ATCC 12600, Staphylococcus aureus ATCC 6358, Staphylococcus aureus MRSA ATCC 33591, E. coli ATCC 11775, P. aeruginosa type 2 mucoid ATCC 33468. The following six reagents were obtained through the National Institutes of Health's National Institute for Allergy and Infectious Disease (NIH/NIAID)'s Biodefense and Emerging Infections Research Resources Repository (BEI Resources): Staphylococcus aureus, Strain AIS2006045 AKA VRS7, NR-46417; Acinetobacter baumannii AB5075-UW, NR49900; Pseudomonas aeruginosa, P179, NR-31041; Salmonella enterica Serovar Typhimurium, Isolate S5682, NR-22068; E. coli serotype O157:H7 ATCC 51657, NR-8; Candida auris AR-0381/CAU-01.

Antibiotic and Antibiofilm Susceptibility Tests. Minimum inhibitory concentration (MIC) broth microdilution method was performed according to CLSI guidelines (CLSI M07-A10: Methods for Dilution Antimicrobial Susceptibility Tests for Bacteria that Grow Aerobically; Approved Standard — Tenth Edition). Minimum bactericidal concentration (MBC) was performed according to CLSI guidelines (CLSI M26-A: Methods for Determining Bactericidal Activity of Antimicrobial Agents; Approved Guideline).

Fractional inhibitory concentration index (FIC) was used to determine the efficacy of the various combinations. The FIC index was determined according to the following equation: FIC Index: [A]/MICA +[B]/MICB, where MICA and MICB are the MICS of each compound alone and [A] and [B] are the MICS of the compounds when used in combination. FIC index: ≤0.5: synergism; >0.5-1: additive; >1-<4: indifferent; ≥4: antagonistic.

The antibiotic resistance breakpoints were defined according to the recommendations of the Clinical and Laboratory Standards Institute (CLSI M07-A10: Methods for Dilution Antimicrobial Susceptibility Tests for Bacteria That Grow Aerobically; Approved Standard— Tenth Edition).

Minimum biofilm eradicating concentration (MBEC) was performed according to the ASTM standard (ASTM E2799-12; Standard Test Method for Testing Disinfectant Efficacy against Pseudomonas aeruginosa Biofilm using the MBEC Assay). Assays were performed in quadruplicates on more than one occasion.

Imaging. Biofilm inhibition imaging was determined by growing biofilm in a black-walled optically-clear bottom 96 well plate at a 45° angle to promote biofilm formation prior to imaging. Biofilm was stained with Syto 9 green fluorescent live and dead nucleic acid stain and measuring total intensity with excitation/emission (nm): 456/541. Images of biofilm were taken using MiniMax 300 Imaging Cytometer (Molecular Devices).

### Example 1. Antibacterial Characteristics of the Organophosphorous and/or Organosulfurous Compounds Alone and in Combination with Antibiotics

Four antibiotics (penicillin G, methicillin, gentamycin, and vancomycin) were tested in combination with SDBS and DBSA, by using a checkerboard methodology against MRSA ATCC 33591 (methicillin-R, gentamicin-R; penicillin-R) and VRSA VRS7 BEI NR-46417 (methicillin-R, gentamicin-R; penicillin-R, vancomycin-R). The combinations were assessed by fractional inhibitory concentration indices (FICIs) and the fold reduction of the MIC (“potentiation factor”) was calculated. In clinical terms, a potentiator enhances the action of a drug. SDBS and SDS potentiate methicillin, gentamicin, penicillin, and vancomycin activity against MRSA and VRSA making MRSA and VRSA sensitive to those antibiotics (Table 13). Synergy and additive effects were observed in the combinations at sub-MIC concentrations of SDBS and DBSA (Table 14).

In combination with SDBS or DBSA, the MIC of gentamicin against VRSA NR-46417 clinical isolate was reduced over 64-fold to 0.33 μg/ml in the presence of 3 μg/ml of SDBS and 8μg/ml of DBSA. In combination with SDBS, the MIC of gentamicin against MRSA ATCC 33591was reduced over 128-fold to 0.33 μg/ml in the presence of 8 μg/ml of SDBS. The MIC of vancomycin against VRSA NR-46417 clinical isolate, in combination with SDBS or DBSA, was reduced over 128-fold to 0.16 μg/ml in the presence of 10 μg/ml of SDBS and 32-fold to 0.33 μg/ml in the presence of 14 μg/ml of DBSA. The MIC of penicillin against VRSA NR-46417 clinical isolate, in combination with SDBS or DBSA, was reduced over 64-fold to 0.16 μg/ml in the presence of 5 μg/ml of SDBS and 10 μg/ml of DBSA. In combination with SDBS, the MIC of penicillin against MRSA ATCC 33591 was reduced over 128-fold to 0.16 μg/ml in the presence of 9 μg/ml of SDBS. In combination with SDBS, the MIC of methicillin against VRSA NR-46417 clinical isolate and MRSA ATCC 33591 was reduced over 128-fold to 0.16 μg/ml in the presence of 10 μg/ml and 16 μg/ml of SDBS, respectively (Table 13). These MIC values are below the CLSI breakpoints for susceptibility of Staphylococcus spp. to these agents.

TABLE 13
Minimum inhibitory concentration (MIC) of sodium
dodecylbenzenesulfonate (SDBS) and 4-Dodecylbenzenesulfonic
acid (DBSA) alone and in combination with existing antibiotics
(methicillin, gentamicin, penicillin, and vancomycin) against
Staphylococcus aureus MRSA (ATCC 33591) and VRSA
(NR-46417).

| | Minimum Inhibitory Concentration | | | |
|---|---|---|---|---|
| | (MIC) (μg/ml) | | | |
| | MRSA | | VRSA VRS7 | |
| | ATCC 33591 | | NR-46417 | |
| SDBS | 10 | | 16 | |
| DBSA | 19 | | >20 | |
| Methicillin | >22.7 | (R) | >22.7 | (R) |
| Methicillin (+ SDBS*) | <0.16 | (S) | <0.16 | (S) |
| Gentamicin | >45.4 | (R) | 22.7 | (R) |
| Gentamicin (+ SDBS*) | 0.33 | (S) | <0.33 | (S) |
| Gentamicin (+ DBSA*) | 1.3 | (S) | <0.33 | (S) |
| Penicillin | >22.7 | (R) | 11.35 | (R) |
| Penicillin (+ SDBS*) | <0.16 | (S) | <0.16 | (S) |

| | | | |
|---|---|---|---|
| Penicillin (+ DBSA*) | — | <0.16 | (S) |
| Vancomycin | — | 22.7 | (R) |
| Vancomycin (+ SDBS*) | — | <0.16 | (S) |
| Vancomycin (+ DBSA*) | — | 0.33 | (S) |

R, resistant;
S, sensitive
*SDBS and DBSA at sub-MIC concentration

TABLE 14
The fractional inhibitory concentration (FIC) index from
combination assay of sodium dodecylbenzenesulfonate (SDBS)
and 4-Dodecylbenzenesulfonic acid (DBSA) in combination
with existing antibiotics (methicillin, gentamicin, penicillin, and
vancomycin) against Staphylococcus aureus MRSA (ATCC 33591)
and VRSA (NR-46417).

| | MRSA | | | VRSA | |
|---|---|---|---|---|---|
| | ATCC 33591 | | | NR-46417 | |
| | FIC Index | Effect | | FIC Index | Effect |
| Gentamicin + | 0.81 | ADD | Gentamicin + | 0.20 | SYN |
| SDBS 8 μg/ml | | | SDBS 3 μg/ml | | |
| Gentamicin + | 0.50 | SYN | Gentamicin + | 0.41 | SYN |
| DBSA 9 μg/ml | | | DBSA 8 μg/ml | | |
| Penicillin + | 0.90 | ADD | Penicillin + | 0.33 | SYN |
| SDBS 9 μg/ml | | | SDBS 5 μg/ml | | |
| Penicillin + | — | — | Penicillin + | 0.51 | SYN |
| DBSA 10 μg/ml | | | DBSA 10 μg/ml | | |
| Methicillin + | 1.61 | IND | Methicillin + | 0.63 | ADD |
| SDBS 16 μg/ml | | | SDBS 10 μg/ml | | |
| Methicillin + | 0.76 | ADD | Methicillin + | — | — |
| DBSA 10 μg/ml | | | DBSA 10 μg/ml | | |
| Vancomycin + | — | — | Vancomycin + | 0.63 | ADD |
| SDBS 10 μg/ml | | | SDBS 10 μg/ml | | |
| Vancomycin + | — | — | Vancomycin + | 0.76 | ADD |
| DBSA 10 μg/ml | | | DBSA 15 μg/ml | | |

The FIC index was determined according to the following equation: FIC Index: [A]/MICA + [B]/MIC_{B}, where MIC_{A} and MIC_{B} are the MICs of each compound alone and [A] and [B] are the MICs of the compounds when used in combination.
FIC index: ≤0.5: synergism (SYN); >0.5-1: additive (ADD); >1-< 4: indifferent (IND); ≥4: antagonistic (ANT).

### Example 2. Antibacterial Activity of the Organophosphorous and/or Organosulfurous Compounds Against Gram-Positive and Gram-Negative Bacteria and Fungi

BPA is active against Staphylococcus aureus MRSA (MIC, 25 μg/ml), S. aureus VRSA (MIC, 25 μg/ml), Pseudomonas aeruginosa type 2 mucoid mutant associated with Cystic Fibrosis (MIC, 25 μg/ml); P. aeruginosa P179 gentamycin-R, streptomycin-R, sulfonamide-R, ampicillin-R, amoxicillin-R, metal ion-R (MIC, 37.5 μg/ml), E. coli serotype O157:H7 streptomycin-R, sulfisoxazole-R, tetracycline-R (MIC, 37.5 μg/ml); Acinetobacter baumannii (MIC, 50 μg/ml); Salmonella enterica serovar Typhimurium ampicillin-R, chloramphenicol-R, kanamycin-R, sulfa-trimethoprim-R, triple sulfa-R, streptomycin-R, tetracycline-R, ceftazidime-R (MIC, 50 μg/ml); Burkholderia cepacia (MIC, 50 μg/ml), and multi-drug resistant Candida auris (MIC, 50 μg/ml). Table 8 provides the minimum inhibitory concentration of BPA against Gram-positive and Gram-negative bacteria.

TABLE 15
Minimum inhibitory concentration (MIC) of butyl phosphoramidate
(BPA) against Gram-positive and Gram-negative bacteria, and fungi,
including drug-resistant strains.

| | BPA |
|---|---|
| | Minimum Inhibitory |
| | Concentration (MIC) |
| | (μg/ml) |
| S. aureus MRSA ATCC 33591 | 12.5 |
| S. aureus VRSA NR46417 | 12.5 |
| P. aeruginosa type 2 mucoid ATCC 33468 | 12.5 |
| P. aeruginosa P179 NR-31041 | 12.5 |
| E. coli serotype O157:H7 ATCC 51657 | 12.5 |
| A. baumannii AB5075-UW, NR49900 | 12.5 |
| Salmonella enterica serovar Typhimurium | 12.5 |
| Isolate S5682, NR-22068 | |
| B. cepacia | 12.5 |
| C. auris AR-0381/CAU-01 | 12.5 |

### Example 3. Antibiofilm Activity of the Organophosphorous and/or Organosulfurous Compounds

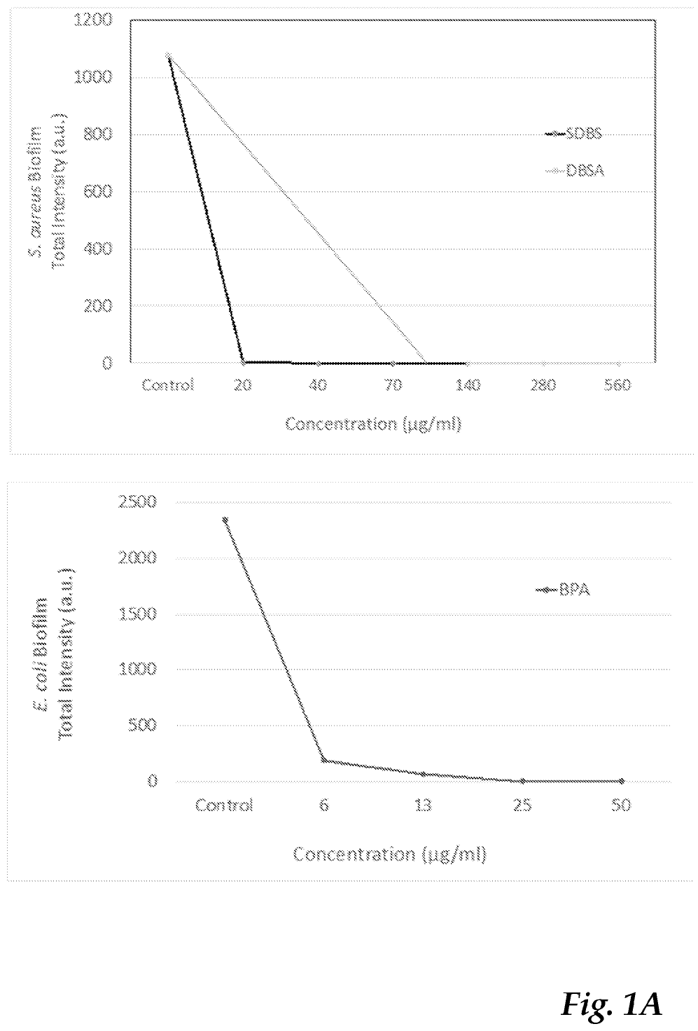

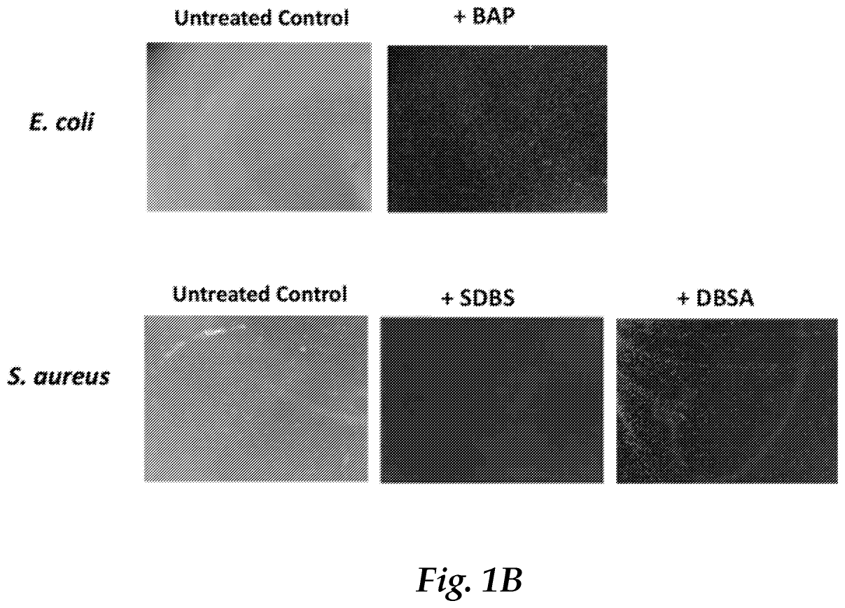

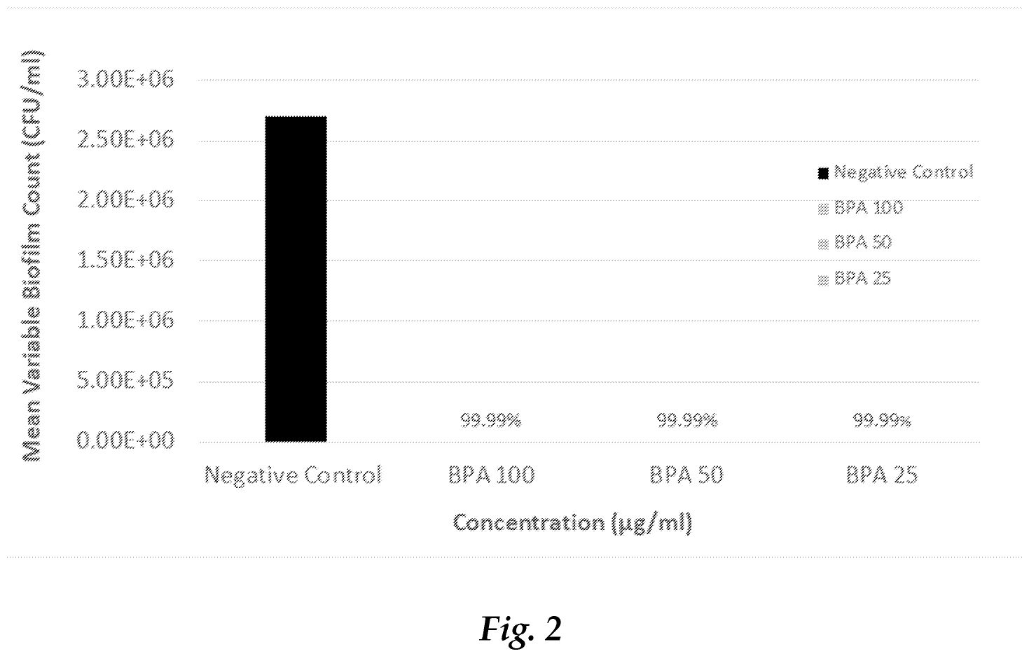

BPA, SDBS, SDS, and DBSA inhibited biofilm formation by S. aureus ATCC 12600 for MBIC concentrations of 50 μg/ml, 20 μg/ml, 140 μg/ml, 100 μg/ml, respectively (FIG. 1). BPA inhibited biofilm formation by E. coli ATCC 11775 for a MBIC concentration of 50 μg/ml (FIG. 1). It was demonstrated by Lonza that BPA removed existing S. aureus ATCC 6358 biofilm for a MBEC concentration <25 μg/ml (FIG. 2). BPA and SDBS inhibited biofilm formation of B. cepacia for MBIC concentrations of 50 μg/ml and 70 μg/ml, respectively.

The organophosphorous and/or organosulfurous compounds alone and in combination with antibiotics described herein are active against MRSA, VRSA, and other Gram-negative bacteria from the WHO Global Priority list including, A. baumanni carbapenem-R and S. enterica fluoroquinolone-R. As well as, P. aeruginosa type 2 mucoid mutant associated with cystic fibrosis, multi-drug resistant P. aeruginosa, multi-drug resistant E. coli, and multi-drug resistant C. auris. They have applications in medical industries to reduce contamination and infection. The organophosphorous and/or organosulfurous compounds have synergistic activity in combination with existing antibiotics and potentiate methicillin, gentamicin, penicillin, and vancomycin activity against MRSA and VRSA, making MRSA and VRSA sensitive to existing antibiotics. The organophosphorous and/or organosulfurous compounds and antibiotic combinations described herein target all modes of microbial growth including biofilms. The organophosphorous and/or organosulfurous compounds prevent biofilm formation and rapidly remove existing Staphylococcus aureus biofilm (within 10 minutes). β-lactamase inhibitors and PBP inhibitors combat resistance by preventing bacterial degradation of β-lactam antibiotics and extending the range of bacteria the antibiotics are effective against.

The compositions described herein can be used to treat and prevent various microbial infections described herein. The compositions described herein can be used as a topical therapeutic for wounds including acute and chronic wounds and pressure wounds, diabetes, chemotherapy, dialysis, and injection sites.

A combination treatment in the form of a topical therapeutic offers unique value added because of the large and growing incidence of AMR Staphylococcus aureus skin and soft tissue infections in all countries and age groups, and particularly in countries with fast-evolving and genetically diverse and resistant strains. Use of an antibacterial and antibiofilm and β-lactamase inhibitors/PBP inhibitors, and silver products can enable patient-based care at home to block contagion, control the emergence of rapidly-evolving AMR S. aureus strains, reduces post-surgical infection, and reduce the burden on healthcare resources (doctor visits, last-resort IV administration, extended hospital stays, etc.). Additionally, the broad spectrum of activity can address other pathogens present in Staphylococcus aureus wounds and infections.

The compositions described herein can be used as a prophylactic against infections on devices, for example, implants, prosthetics, instruments including instruments for sutures, catheters, and respirators. A combination treatment in a device coating that integrates multiple antimicrobial and/or antibiofilm effects that are used to treat and prevent infection has the potential to reduce hospital-acquired infections and diseases.

The compositions described herein can be used as a wash, for example at wounds and burn sites. The compositions can be used as a pre-surgical and postsurgical wash or as a presurgical nasal swab. The compositions described herein also can be used as surface cleaners to clean any surface for environmental sanitation. In embodiments, the compositions are used as surface cleaners in hospitals, for example, prior to all clinical procedures.

A prebiotic and probiotic may be combined with antimicrobial topical treatments to accelerate skin regeneration. A prebiotic and probiotic may be combined with other antimicrobials to minimize the impact of treatment on beneficial gut flora.

All publications and patent applications herein are incorporated by reference to the same extent as if each individual publication or patent application was specifically and individually indicated to be incorporated by reference.

1-22. (canceled)

23. A method of preventing the growth of or removing or killing one or more microorganisms from on a clinical surface, the method comprising contacting the clinical surface with a composition comprising an effective amount of a composition comprising one or more of organophosphorous or organosulfurous compounds, or salts, acids or bases thereof, and one or more of antimicrobials, antimicrobial potentiators, probiotics, or prebiotics, wherein the one or more of organophosphorous or organosulfurous compounds having have the structure of: 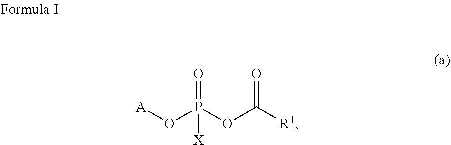
wherein A is C₁₋₁₀ hydrocarbyl or C₁₋₁₀ hydrocarbyl substituted with R¹; X is NHR, NHOR, NHCOR, NHOCOR, or OR; and R is H or C₁₋₁₀ hydrocarbyl;
R¹ is hydrogen, halogen, cyano, OH, C₁₋₆ hydrocarbyl, C₁₋₆ alkoxy, SOR², SO₂R², SO₂NR³R⁴COR², CO₂R², CONR³R⁴, NR³R⁴, NR³COR⁴, NR³SO₂R⁴, NR³CO₂R⁴, NR³CONR⁴, OCOR², or phosphonic acid, wherein each of C₁₋₆ hydrocarbyl, C₁₋₆ alkoxy, SOR², SO₂R², SO₂NR³R⁴, COR², CO₂R², CONR³R⁴, NR³R⁴, NR³COR⁴, NR³SO₂R⁴, NR³CO₂R⁴, NR³CONR⁴, or OCOR², can be optionally substituted with halo, amino, hydroxyl, C₁₋₆ hydrocarbyl,
C₁₋₆ alkoxy, cyano, or phosphonic acid; and
R², R³, and R⁴ are independently selected from hydrogen or C₁₋₆ hydrocarbyl, wherein each of the C₁₋₆ hydrocarbyls can be optionally substituted with halo, amino, hydroxyl, C₁₋₆ alkoxy, cyano, or phosphonic acid;
(b) 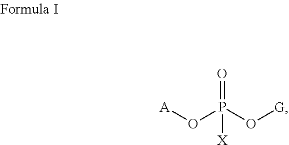
wherein A is R; Y is O or S; X is NH₂, OH, or OR; and G is R, wherein R is C₁₋₁₆ hydrocarbyl; 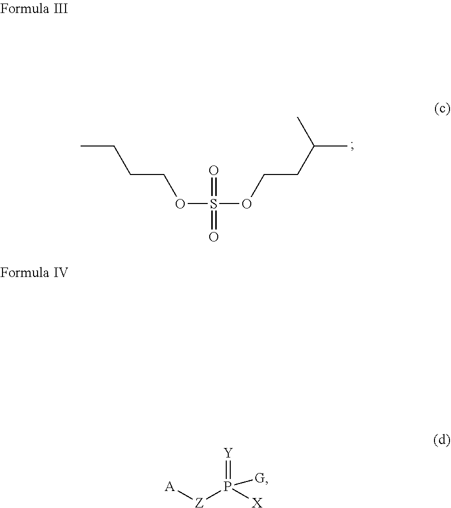
wherein A is H, C₁₋₁₆ hydrocarbyl or C₁₋₁₆ hydrocarbyl substituted with R¹; Z is O or a bond; Y is O; G is OH, H, C₁₋₆—COO-alkyl, O—C₁₋₁₆ alkyl, C₁₋₁₆ hydrocarbyl, R¹ substituted C₁₋₁₆ hydrocarbyl, H₂NHCH₂COOH, or O-aryl; X is H, CN, —NHR, —NHOR, —NHOCOR, hydrocarbyl, R¹ substituted C₁₋₁₀ hydrocarbyl, or —OR; and R is H, hydrocarbyl, or R¹ substituted C₁₋₁₀ hydrocarbyl;
R¹ is halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, or phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic acid; and R², R³, and R⁴ are independently selected from hydrogen, C₁₋₆ hydrocarbyl, in which each of the C₁₋₆ hydrocarbyls can be optionally substituted with halo, amino, hydroxyl, C₁₋₆ alkoxy, cyano, or phosphonic acid; or
(e) 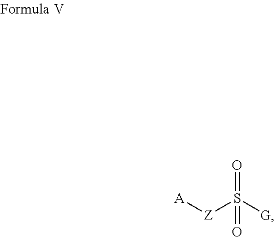
wherein A is H, C₁₋₂₀ hydrocarbyl, alkylaryl, or C₁₋₂₀ hydrocarbyl substituted with R¹; Z is O or a bond; G is OH, H, C₁₋₆—COO-alkyl, O—C₁₋₆ alkyl, C₁₋₁₆ hydrocarbyl, R¹ substituted C₁₋₁₆ hydrocarbyl, H₂NHCH₂COOH, or O-aryl;
R¹ is halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, or phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic acid; and
R², R³, and R⁴ are independently selected from hydrogen, C₁₋₆ hydrocarbyl, in which each of the C₁₋₆ hydrocarbyls can be optionally substituted with halo, amino, hydroxyl, C₁₋₆ alkoxy, cyano, or phosphonic acid.

24. The method of claim 23, wherein the one or more oganophosphorous or organosulfurous compounds, or salts, acids, or bases thereof, have the structure shown in Table 1, 2, 3, 4, 5, 6, 7, 8, 9, 10, 11, or 12 or are represented by

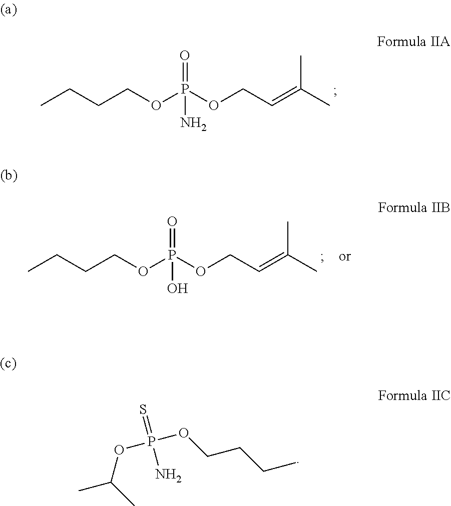

25. The method of claim 23, wherein the composition comprises an antiseptic, a cleaning agent, a dispersant, a surfactant, an anti-odor agent, an anti-biofilm agent, an anti-fouling agent, an antimicrobial, a therapeutic agent, a prophylactic antibiotic, or a combination thereof.

26. The method of claim 23, wherein the one or more microorganisms are pathogenic to humans, animals, other living organisms, or a combination thereof.

27. The method of claim 23, wherein the one or more microorganisms comprises bacteria, fungi, viruses, or a combination thereof.

28. The method of claim 23, wherein the one or more microorganisms comprises Staphylococcus aureus, Staphylococcus epidermidis, Acinetobacter baumannii, Pseudomonas aeruginosa, Enterobacteriaceae, Escherichia coli, Salmonella enterica, Salmonella bongori, Enterococcus faecium, Helicobacter pylori, Campylobacter spp., Neisseria gonorrhoeae, Streptococcus pneumoniae, Streptococcus mutans, Streptococcus gordonii, Streptococcus pyogenes, Haemophilus influenzae, Shigella spp., Klebsiella pneumoniae, Clostridium difficile, Bacillus anthracis, Yersinia pestis, Francisella tularensis, Burkholderia mallei, Burkholderia pseudomallei, Corynebacterium spp., Micrococcus luteus, Micrococcus lylae, Micrococcus roseus, Cutibaceterium acnes, Vibrio vulnificus, Vibrio cholerae, Vibrio parahaemolyticus, Propionibacterium acnes, Neisseria gonorrhoeae, Burkholderia cepacia, Burkholderia mallei, Burkholderia pseudomallei, Ralstonia pickettii, Cuprividus metallidurans, or a combination thereof.

29. The method of claim 23, wherein the one or more microorganisms comprises Candida albicans, Candida auris, Aspergillus fumigatus, Aspergillus flavus, Apohysomyces sp., Blastomyces dermatitidis, Coccidioides posadasii, Cryptococcus neoformans, Fusarium spp., Histoplasma capsulatum, Pneumocystis jirovecii, or Rhizopus oryzae, Scedosporium spp., or a combination thereof.

30. The method of claim 23, wherein the one or more microorganisms are methicillin-sensitive, methicillin-resistant, vancomycin-intermediate, vancomycin-resistant, carbapenem-resistant, clarithromycin-resistant, cephalosporin-resistant, am picillin-resistant, penicillin-non-susceptible, fluconazole-resistant, am photericin-resistant, echinocandins-resistant, m upirocin-resistant, erythromycin-resistant, streptomycin-resistant, tetracycline-resistant, amoxicillin-resistant, ciprofloxacin-resistant, silver-resistant, salicylic acid-resistant, neomycin sulfate-resistant, polymyxin B sulfate-resistant, bacitracin-resistant, zinc-resistant, alcohol-tolerant, alcohol-resistant, chlorhexidine-resistant, fluoroquinolone-resistant, or a combination thereof.

31. The method of claim 23, wherein the one or more organophosphorous or organosulfurous compounds, or salts, acids, or bases thereof comprises sodium dodecylbenzenesulfonate (SDBS), sodium dodecyl sulfate (SDS), 4-Dodecylbenzenesulfonic acid (DBSA), butyl phosphoramidate (BPA), (4-aminophenethyl)dimethylphosphine oxide (APDMPO), or a combination thereof.

32. The method of claim 23, wherein the composition further comprises one or more antimicrobials and optionally wherein the antimicrobial is a β-lactam, aminoglycoside, glycopeptide, macrolide, fluoroquinolone, sulfonamide, tetracycline, mupirocin, salicylate, polymixin, butenafine hydrochloride, clotrimazole, miconazole nitrate, terbinafine hydrochloride, fluoroquinolone carboxylic acid derivatives, tryptophan, thiosulfil, plazomicin, fosfomycin, cefepime, maxipime, pravibismane, tolnaftate, piperazines, bismuth thiols, bismuth thiol complexes, bismuth dithiol complexes, chlorhexidine, itaconic acid, colistin, potassium carbonate, isothiocyanates, phenyl isothiocyanate, sodium carbonate, sodium bicarbonate, calcium phosphate, calcium carbonate, methyl ethyl ketone, iodine, povidone-iodine, acetic acid, hydrogen peroxide, peroxide, peracetic acid, sodium hypochlorite, or a combination thereof.

33. The method of 23, wherein the composition further comprises one or more silver compounds, antibiotic adjuvants, non-steroidal anti-inflammatory drugs, protein kinase inhibitors, quorum sensing signal inhibitors, or a combination thereof.

34. The method of claim 33, wherein the one or more silver compounds comprise a silver ion, silver particle, silver nanoparticle, metallic silver, colloidal silver, silver chloride, or a combination thereof;
wherein the one or more antibiotic adjuvants comprise a β-lactamase inhibitor, penicillin-binding protein inhibitor, dihydropteroate synthetase inhibitor, iron chelator, or a combination thereof;
wherein the one or more non-steroidal anti-inflammatory drugs comprise aspirin;
wherein the protein kinase inhibitor comprises serine/threonine kinase inhibitor, tyrosine kinase inhibitor, or serine/threonine/tyrosine kinase inhibitor; and
wherein the quorum sensing signal inhibitor comprises Avellanin C, p-nitrophenyl glycerol, tannic acid, or RNAIII inhibiting peptide.

35-41. (canceled)

42. The method of claim 23, wherein the composition further comprises a microbially produced metabolite, enzyme, bacteriocin, and/or acid.

43. The method of claim 42, wherein the microbially produced metabolite is propionate, butyrate, acetate, serine endopeptidase, succinic acid, lactic acid, formic acid, propionic acid, nisin, actagardine, durancin 61A, and/or PsVP-10.

44. A method of treating or preventing a disease or condition caused by one or more microorganisms in a subject, the method comprising administrating an effective amount of a composition comprising one or more of organophosphorous or organosulfurous compounds, or salts, acids, or bases thereof having the structure of:
(a) 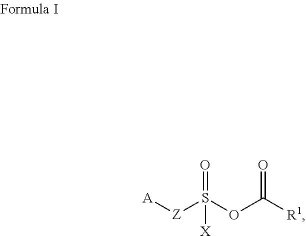
wherein A is C₁₋₁₀ hydrocarbyl or C₁₋₁₀ hydrocarbyl substituted with R¹; X is NHR, NHOR, NHCOR, NHOCOR, or OR; and R is H or C₁₋₁₀ hydrocarbyl;
R¹ is hydrogen, halogen, cyano, OH, C₁₋₆ hydrocarbyl, C₁₋₆ alkoxy, SOR², SO₂R², SO₂NR³R⁴COR², CO₂R², CONR³R⁴, NR³R⁴, NR³COR⁴, NR³SO₂R⁴, NR³CO₂R⁴, NR³CONR⁴, OCOR², or phosphonic acid, wherein each of C₁₋₆ hydrocarbyl, C₁₋₆ alkoxy, SOR², SO₂R², SO₂NR³R⁴, COR², CO₂R², CONR³R⁴, NR³R⁴, NR³COR⁴, NR³SO₂R⁴, NR³CO₂R⁴, NR³CONR⁴, or OCOR², can be optionally substituted with halo, amino, hydroxyl, C₁₋₆ hydrocarbyl,
C₁₋₆ alkoxy, cyano, or phosphonic acid; and
R², R³, and R⁴ are independently selected from hydrogen or C₁₋₆ hydrocarbyl, wherein each of the C₁₋₆ hydrocarbyls can be optionally substituted with halo, amino, hydroxyl, C₁₋₆ alkoxy, cyano, or phosphonic acid; 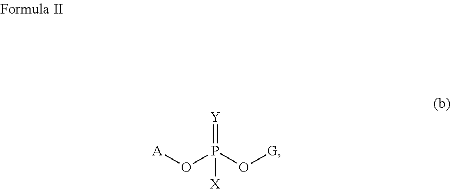
wherein A is R; Y is O or S; X is NH₂, OH, or OR; and G is R, wherein R is C₁₋₁₆ hydrocarbyl; 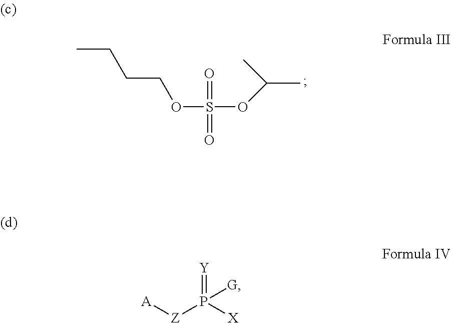
wherein A is H, C₁₋₁₆ hydrocarbyl or C₁₋₁₆ hydrocarbyl substituted with R¹; Z is O or a bond; Y is O; G is OH, H, C₁₋₆—COO-alkyl, O—C₁₋₁₆ alkyl, C₁₋₁₆ hydrocarbyl, R¹ substituted C₁₋₁₆ hydrocarbyl, CH₂NHCH₂COOH, or O-aryl; X is H, CN, —NHR, —NHOR, —NHOCOR, hydrocarbyl, R¹ substituted C₁₋₁₀ hydrocarbyl or —OR; and R is H, hydrocarbyl, or R¹ substituted C₁₋₁₀ hydrocarbyl;
R¹ is halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, or phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic acid; and
R², R³, and R⁴ are independently selected from hydrogen, C₁₋₆ hydrocarbyl, in which each of the C₁₋₆ hydrocarbyls can be optionally substituted with halo, amino, hydroxyl, C₁₋₆ alkoxy, cyano, or phosphonic acid; or 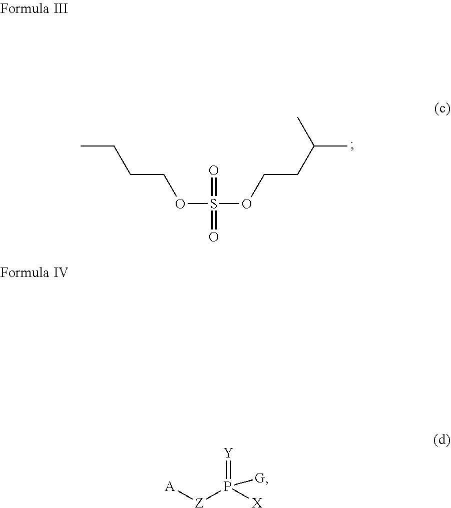
wherein A is H, C₁₋₂₀ hydrocarbyl, alkylaryl, or C₁₋₂₀ hydrocarbyl substituted with R¹; Z is O or a bond; G is OH, H, C₁₋₆—COO-alkyl, O—C₁₋₆ alkyl, C₁₋₁₆ hydrocarbyl, R¹ substituted C₁₋₁₆ hydrocarbyl, CH₂NHCH₂COOH, or O-aryl;
R¹ is halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, or phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic acid; and
R², R³, and R⁴ are independently selected from hydrogen, C₁₋₆ hydrocarbyl, in which each of the C₁₋₆ hydrocarbyls can be optionally substituted with halo, amino, hydroxyl, C₁₋₆ alkoxy, cyano, or phosphonic acid.

45. The method of claim 44, wherein the one or more oganophosphorous or organosulfurous compounds, or salts, acids or bases thereof have the structure shown in Table 1, 2, 3, 4, 5, 6, 7, 8, 9, 10, 11, or 12 or have the structure of:

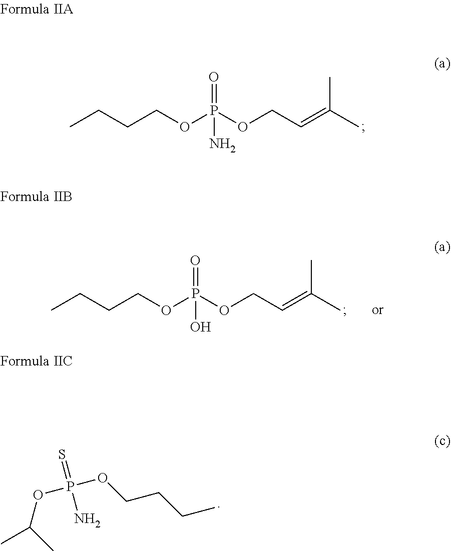

46. The method of claim 44, wherein the composition further comprises one or more antimicrobials, antimicrobial potentiators, or prebiotics.

47. The method of claim 44, wherein the composition is administered topically, orally, intravenously, or aerosolized.

48. The method of claim 44, wherein the one or more microorganisms are bacteria, fungi, viruses, or a combination thereof.

49. The method of claim 44, wherein the one or more microorganisms are methicillin-sensitive, methicillin-resistant, vancomycin-intermediate, vancomycin-resistant, carbapenem-resistant, clarithromycin-resistant, cephalosporin-resistant, ampicillin-resistant, penicillin-non-susceptible, fluconazole-resistant, am photericin-resistant, echinocandins-resistant, mupirocin-resistant, erythromycin-resistant, streptomycin-resistant, tetracycline-resistant, amoxicillin-resistant, ciprofloxacin-resistant, silver-resistant, salicylic acid-resistant, neomycin sulfate-resistant, polymyxin B sulfate-resistant, bacitracin-resistant, zinc-resistant, alcohol-tolerant, alcohol-resistant, chlorhexidine-resistant, fluoroquinolone-resistant, or a combination thereof.
